(19) United States
(12) Patent Application Publication
(10) Pub. No.: US 2026/0285976 A1
(43) Pub. Date: Sep. 24, 2026

(54) ANTIBODIES BINDING TO LY6G6D

(71) Applicant: QLSF Biotherapeutics, Inc., South San Francisco, CA (US)

(72) Inventors: Shenda GU, South San Francisco, CA (US); Shihao CHEN, Souith San Francisco, CA (US)

(21) Appl. No.: 19/475,729

(22) PCT Filed: Apr. 5, 2024
(86) PCT No.: PCT/US2024/023363

§ 371 Date: Oct. 15, 2025

Related U.S. Application Data
(60) Provisional application No. 63/496,928, filed on Apr. 18, 2023.

(51) Int. Cl. C07K 16/28; A61K 39/00; A61P 35/00

(52) U.S. Cl. CPC C07K 16/2809; A61P 35/00; A61K 2039/505; C07K 2317/31; C07K 2317/565; C07K 2317/73; C07K 2317/92

(57) ABSTRACT

Anti-LY6G6D antibodies are disclosed, along with methods of making such antibodies, compositions, including pharmaceutical compositions, comprising such antibodies, and their use to treat disorders that are characterized by the expression of LY6G6D.

### CROSS-REFERENCE TO RELATED APPLICATIONS

This application claims priority benefit of the filing date of U.S. provisional patent application Ser. No. 63/496,928, filed on Apr. 18, 2023, the disclosure of which is incorporated by reference herein in its entirety.

### FIELD OF THE INVENTION

The present invention concerns antibodies binding to LY6G6D. The invention further concerns methods of making such antibodies, compositions, including pharmaceutical compositions, comprising such antibodies, and their use to treat disorders that are characterized by the expression of LY6G6D.

### BACKGROUND OF THE INVENTION

### LY6G6D

LY6G6D belongs to a cluster of leukocyte antigen-6 (LY6) genes located in the major histocompatibility complex (MHC) class III region on chromosome 6. Members of the LY6 superfamily typically contain 70 to 80 amino acids, including 8 to 10 cysteines. Most LY6 proteins are attached to the cell surface by a glycosylphosphatidylinositol (GPI) anchor that is directly involved in signal transduction.

RNA expression analysis from TCGA databases shows that LY6G6D is highly expressed and specific to colorectal cancer. Expression on other tumor types and normal cells is negligible, making it a highly favorable tumor target. The expression of LY6G6D is associated with cancers that do not respond well to immune checkpoint inhibitors. Therapeutic development of antibodies against this target may therefore be efficacious in treating certain cohorts of colorectal cancer patients that are refractory to chemotherapy or immune checkpoint therapy.

### SUMMARY OF THE INVENTION

Aspects of the invention relate to LY6G6D-binding antibodies. Further aspects of the invention relate to methods of making such antibodies, compositions comprising such antibodies, and their use in the treatment of disorders that are characterized by the expression of LY6G6D.

In some embodiments, an antibody that binds to LY6G6D comprises a first binding unit comprising: a heavy chain variable region comprising: (a) a CDRH1 sequence of any one of SEQ ID NOs: 1-6; and/or (b) a CDRH2 sequence of any one of SEQ ID NOs: 7-11; and/or (c) a CDRH3 sequence of any one of SEQ ID NOs: 12-15; and a light chain variable region comprising: (d) a CDRL1 sequence of any one of SEQ ID NOs: 16-19; and/or (e) a CDRL2 sequence of any one of SEQ ID NOs: 20-23; and/or (f) a CDRL3 sequence of any one of SEQ ID NOs: 24-26. In some embodiments, the CDRH1, CDRH2 and CDRH3 sequences of the first binding unit are present in a human VH framework. In some embodiments, CDRL1, CDRL2 and CDRL3 sequences of the first binding unit are present in a human VL framework.

In some embodiments, the first binding unit comprises: a heavy chain variable region comprising: (a) a CDRH1 sequence of any one of SEQ ID NOs: 1-6; and (b) a CDRH2 sequence of any one of SEQ ID NOs: 7-11; and (c) a CDRH3 sequence of any one of SEQ ID NOs: 12-15.

In some embodiments, the first binding unit comprises: a heavy chain variable region comprising: (a) a CDRH1 sequence of SEQ ID NO: 1, a CDRH2 sequence of SEQ ID NO: 7, and a CDRH3 sequence of SEQ ID NO: 12; or (b) a CDRH1 sequence of SEQ ID NO: 2, a CDRH2 sequence of SEQ ID NO: 8, and a CDRH3 sequence of SEQ ID NO: 13; or (c) a CDRH1 sequence of SEQ ID NO: 3, a CDRH2 sequence of SEQ ID NO: 9, and a CDRH3 sequence of SEQ ID NO: 13; or (d) a CDRH1 sequence of SEQ ID NO: 4, a CDRH2 sequence of SEQ ID NO: 10, and a CDRH3 sequence of SEQ ID NO: 14; or (e) a CDRH1 sequence of SEQ ID NO: 5, a CDRH2 sequence of SEQ ID NO: 11, and a CDRH3 sequence of SEQ ID NO: 15; or (f) a CDRH1 sequence of SEQ ID NO: 6, a CDRH2 sequence of SEQ ID NO: 11, and a CDRH3 sequence of SEQ ID NO: 15.

In some embodiments, the first binding unit comprises: a light chain variable region comprising: (a) a CDRL1 sequence of any one of SEQ ID NOs: 16-19; and (b) a CDRL2 sequence of any one of SEQ ID NOs: 20-23; and (c) a CDRL3 sequence of any one of SEQ ID NOs: 24-26.

In some embodiments, the first binding unit comprises: a light chain variable region comprising: (a) a CDRL1 sequence of SEQ ID NO: 16, a CDRL2 sequence of SEQ ID NO: 20, and a CDRL3 sequence of SEQ ID NO: 24; or (b) a CDRL1 sequence of SEQ ID NO: 17, a CDRL2 sequence of SEQ ID NO: 21, and a CDRL3 sequence of SEQ ID NO: 25; or (c) a CDRL1 sequence of SEQ ID NO: 18, a CDRL2 sequence of SEQ ID NO: 22, and a CDRL3 sequence of SEQ ID NO: 24; or (d) a CDRL1 sequence of SEQ ID NO: 19, a CDRL2 sequence of SEQ ID NO: 23, and a CDRL3 sequence of SEQ ID NO: 26.

In some embodiments, the first binding unit comprises: (a) a heavy chain variable region comprising: a CDRH1 sequence of SEQ ID NO: 1, a CDRH2 sequence of SEQ ID NO: 7, and a CDRH3 sequence of SEQ ID NO: 12; and a light chain variable region comprising: a CDRL1 sequence of SEQ ID NO: 16, a CDRL2 sequence of SEQ ID NO: 20, and a CDRL3 sequence of SEQ ID NO: 24; or (b) a heavy chain variable region comprising: a CDRH1 sequence of SEQ ID NO: 2, a CDRH2 sequence of SEQ ID NO: 8, and a CDRH3 sequence of SEQ ID NO: 13; and a light chain variable region comprising: a CDRL1 sequence of SEQ ID NO: 17, a CDRL2 sequence of SEQ ID NO: 21, and a CDRL3 sequence of SEQ ID NO: 25: or (c) a heavy chain variable region comprising: a CDRH1 sequence of SEQ ID NO: 3, a CDRH2 sequence of SEQ ID NO: 9, and a CDRH3 sequence of SEQ ID NO: 13; and a light chain variable region comprising: a CDRL1 sequence of SEQ ID NO: 17, a CDRL2 sequence of SEQ ID NO: 21, and a CDRL3 sequence of SEQ ID NO: 25; or (d) a heavy chain variable region comprising: a CDRH1 sequence of SEQ ID NO: 4, a CDRH2 sequence of SEQ ID NO: 10, and a CDRH3 sequence of SEQ ID NO: 14; and a light chain variable region comprising: a CDRL1 sequence of SEQ ID NO: 18, a CDRL2 sequence of SEQ ID NO: 22, and a CDRL3 sequence of SEQ ID NO: 24; or (e) a heavy chain variable region comprising: a CDRH1 sequence of SEQ ID NO: 5, a CDRH2 sequence of SEQ ID NO: 11, and a CDRH3 sequence of SEQ ID NO: 15; and a light chain variable region comprising: a CDRL1 sequence of SEQ ID NO: 19, a CDRL2 sequence of SEQ ID NO: 23, and a CDRL3 sequence of SEQ ID NO: 26; or (f) a heavy chain variable region comprising: a CDRH1 sequence of SEQ ID NO: 6, a CDRH2 sequence of SEQ ID NO: 11, and a CDRH3 sequence of SEQ ID NO: 15; and a light chain variable region comprising: a CDRL1 sequence of SEQ ID NO: 19, a CDRL2 sequence of SEQ ID NO: 23, and a CDRL3 sequence of SEQ ID NO: 26.

In some embodiments, the first binding unit comprises a heavy chain variable region sequence having at least 95% identity to any one of SEQ ID NOs: 27-34.

In some embodiments, the first binding unit the first binding unit comprises a heavy chain variable region sequence comprising any one of SEQ ID NOs: 27-34.

In some embodiments, the first binding unit comprises a light chain variable region sequence having at least 95% identity to any one of SEQ ID NOs: 35-42.

In some embodiments, the first binding unit comprises a light chain variable region sequence comprising any one of SEQ ID NOs: 35-42.

In some embodiments, the first binding unit comprises: (a) a heavy chain variable region sequence of SEQ ID NO: 27 and a light chain variable region sequence of SEQ ID NO: 35; or (b) a heavy chain variable region sequence of SEQ ID NO: 28 and a light chain variable region sequence of SEQ ID NO: 36; or (c) a heavy chain variable region sequence of SEQ ID NO: 29 and a light chain variable region sequence of SEQ ID NO: 37; or (d) a heavy chain variable region sequence of SEQ ID NO: 30 and a light chain variable region sequence of SEQ ID NO: 38; or (e) a heavy chain variable region sequence of SEQ ID NO: 31 and a light chain variable region sequence of SEQ ID NO: 39; or (l) a heavy chain variable region sequence of SEQ ID NO: 32 and a light chain variable region sequence of SEQ ID NO: 40; or (g) a heavy chain variable region sequence of SEQ ID NO: 33 and a light chain variable region sequence of SEQ ID NO: 41; or (h) a heavy chain variable region sequence of SEQ ID NO: 34 and a light chain variable region sequence of SEQ ID NO: 42.

In some embodiments, an antibody further comprises a heavy chain constant region. In some embodiments, the heavy chain constant region comprises a hinge region, a CH1 region, a CH2 region, and/or a CH3 region. In some embodiments, the heavy chain constant region comprises one or more knob-in-hole (KiH) mutations. In some embodiments, the heavy chain constant region comprises one or more silencing mutations. In some embodiments, the heavy chain constant region comprises one or more FcRn binding mutations. In some embodiments, the one or more FcRn binding mutations comprise a T250Q mutation, an M428L mutation, or both a T250Q and an M428L mutation. In some embodiments, the heavy chain constant region comprises one or more protein A binding mutations. In some embodiments, the one or more protein A binding mutations comprise an H435R mutation, a Y436F mutation, or both an H435R and a Y436F mutation.

In some embodiments, an antibody further comprises a light chain constant region. In some embodiments, the light chain constant region comprises a CL region. In some embodiments, an antibody is monospecific. In some embodiments, an antibody is multispecific. In some embodiments, an antibody is bispecific.

In some embodiments, an antibody further comprises a second binding unit that binds to CD38. In some embodiments, the second binding unit comprises: a heavy chain variable region comprising: (a) a CDRH1 sequence of SEQ ID NO: 43; and/or (b) a CDRH2 sequence of any one of SEQ ID NOs: 44-45; and/or (c) a CDRH3 sequence of SEQ ID NO: 46; and a light chain variable region comprising: (d) a CDRL1 sequence of SEQ ID NO: 47; and/or (e) a CDRL2 sequence of SEQ ID NO: 48; and/or (f) a CDRL3 sequence of SEQ ID NO: 49. In some embodiments, the CDRH1, CDRH2 and CDRH3 sequences of the second binding unit are present in a human VH framework. In some embodiments, the CDRL1, CDRL2 and CDRL3 sequences of the second binding unit are present in a human VL framework. In some embodiments, the second binding unit comprises: a heavy chain variable region comprising: (a) a CDRH1 sequence of SEQ ID NO: 43; and (b) a CDRH2 sequence of any one of SEQ ID NOs: 44-45; and (c) a CDRH3 sequence of SEQ ID NO: 46. In some embodiments, the second binding unit comprises: a heavy chain variable region comprising: (a) a CDRH1 sequence of SEQ ID NO: 43, a heavy chain CDRH2 sequence of SEQ ID NO: 44, and a heavy chain CDRH3 sequence of SEQ ID NO: 46; or (b) a CDRH1 sequence of SEQ ID NO: 43, a CDRH2 sequence of SEQ ID NO: 45, and a CDRH3 sequence of SEQ ID NO: 46.

In some embodiments, the second binding unit comprises: a light chain variable region comprising: a CDRL1 sequence of SEQ ID NO: 47; a CDRL2 sequence of SEQ ID NO: 48; and a CDRL3 sequence of SEQ ID NO: 49. In some embodiments, the second binding unit comprises: (a) a heavy chain variable region comprising: a CDRH1 sequence of SEQ ID NO: 43, a CDRH2 sequence of SEQ ID NO: 44, and a CDRH3 sequence of SEQ ID NO: 46; and a light chain variable region comprising: a CDRL1 sequence of SEQ ID NO: 47, a CDRL2 sequence of SEQ ID NO: 48, and a CDRL3 sequence of SEQ ID NO: 49; or (b) a heavy chain variable region comprising: a CDRH1 sequence of SEQ ID NO: 43, a CDRH2 sequence of SEQ ID NO: 45, and a CDRH3 sequence of SEQ ID NO: 46; and a light chain variable region comprising: a CDRL1 sequence of SEQ ID NO: 47, a CDRL2 sequence of SEQ ID NO: 48, and a CDRL3 sequence of SEQ ID NO: 49.

In some embodiments, the second binding unit comprises a heavy chain variable region sequence having at least 95% identity to any one of SEQ ID NOs: 50-51. In some embodiments, the second binding unit comprises a heavy chain variable region sequence comprising any one of SEQ ID NOs: 50-51. In some embodiments, the second binding unit comprises a light chain variable region sequence having at least 95% identity to any one of SEQ ID NOs: 52-53. In some embodiments, the second binding unit comprises a light chain variable region sequence comprising any one of SEQ ID NOs: 52-53.

In some embodiments, the second binding unit comprises: (a) a heavy chain variable region sequence of SEQ ID NO: 50 and a light chain variable region sequence of SEQ ID NO: 52; or (b) a heavy chain variable region sequence of SEQ ID NO: 51 and a light chain variable region sequence of SEQ ID NO: 53.

Aspects of the invention include an antibody that binds to LY6G6D and CD3ε, comprising: a first light chain subunit comprising SEQ ID NO: 57; a first heavy chain subunit comprising SEQ ID NO: 54; a second light chain subunit comprising SEQ ID NO: 57; and a second heavy chain subunit comprising SEQ ID NO: 56.

Aspects of the invention include an antibody that binds to LY6G6D and CD3ε, comprising: a first light chain subunit comprising SEQ ID NO: 57; a first heavy chain subunit comprising SEQ ID NO: 55; a second light chain subunit comprising SEQ ID NO: 57; and a second heavy chain subunit comprising SEQ ID NO: 56.

Aspects of the invention include an antibody that binds to LY6G6D and CD3ε, comprising: a first light chain subunit comprising SEQ ID NO: 57; a first heavy chain subunit comprising SEQ ID NO: 58; a second light chain subunit comprising SEQ ID NO: 57; and a second heavy chain subunit comprising SEQ ID NO: 60.

Aspects of the invention include an antibody that binds to LY6G6D and CD3ε, comprising: a first light chain subunit comprising SEQ ID NO: 57; a first heavy chain subunit comprising SEQ ID NO: 59; a second light chain subunit comprising SEQ ID NO: 57; and a second heavy chain subunit comprising SEQ ID NO: 60.

Aspects of the invention include an antibody that binds to LY6G6D and CD3ε, comprising: a first light chain subunit comprising SEQ ID NO: 57; a first heavy chain subunit comprising SEQ ID NO: 61; a second light chain subunit comprising SEQ ID NO: 57; and a second heavy chain subunit comprising SEQ ID NO: 62.

Aspects of the invention include an antibody that binds to LY6G6D and CD3ε, comprising: a first light chain subunit comprising SEQ ID NO: 69; a first heavy chain subunit comprising SEQ ID NO: 64; a second light chain subunit comprising SEQ ID NO: 69; and a second heavy chain subunit comprising SEQ ID NO: 66.

Aspects of the invention include an antibody that binds to LY6G6D and CD3ε, comprising: a first light chain subunit comprising SEQ ID NO: 69; a first heavy chain subunit comprising SEQ ID NO: 65; a second light chain subunit comprising SEQ ID NO: 69; and a second heavy chain subunit comprising SEQ ID NO: 66.

Aspects of the invention include an antibody that binds to LY6G6D and CD3ε, comprising: a first light chain subunit comprising SEQ ID NO: 69; a first heavy chain subunit comprising SEQ ID NO: 67; a second light chain subunit comprising SEQ ID NO: 69; and a second heavy chain subunit comprising SEQ ID NO: 68.

Aspects of the invention include pharmaceutical compositions comprising an antibody as described herein.

Aspects of the invention include methods of treatment, comprising administering to an individual in need an effective dose of an antibody or pharmaceutical composition as described herein.

Aspects of the invention include methods for the treatment of a disorder characterized by expression of LY6G6D, comprising administering to a subject with said disorder an antibody or a pharmaceutical composition as described herein.

Aspects of the invention include use of an antibody as described herein, in the preparation of a medicament for the treatment of a disorder characterized by expression of LY6G6D.

Aspects of the invention include an antibody as described herein, for use in the treatment of a disorder characterized by expression of LY6G6D.

In some embodiments, the disorder is a cancer. In some embodiments, the cancer is a solid tumor cancer. In some embodiments, the solid tumor cancer is colectoral cancer. In some embodiments, the colectoral cancer is refractive to chemotherapy and/or immune checkpoint inhibitor therapy.

Aspects of the invention include a polynucleotide encoding an antibody as described herein, a vector comprising such a polynucleotide, and a cell comprising such a vector.

Aspects of the invention include methods of producing an antibody as described herein, comprising growing a cell as described herein under conditions permissive for expression of the antibody, and isolating the antibody.

Aspects of the invention include kits comprising an antibody or a pharmaceutical composition as described herein, and instructions for use. In some embodiments, a kit further comprises an additional therapeutic agent. In some embodiments, the additional therapeutic agent comprises a chemotherapeutic drug.

Aspects of the invention include diagnostic methods for determining whether a subject has or is at risk of developing a disorder characterized by expression of LY6G6D, the methods comprising: contacting a biological test sample from the subject with an antibody that binds to LY6G6D to generate an LY6G6D-antibody complex, wherein the antibody comprises: (a) a heavy chain variable region comprising: a CDRH1 sequence of SEQ ID NO: 1. a CDRH2 sequence of SEQ ID NO: 7, and a CDRH3 sequence of SEQ ID NO: 12; and a light chain variable region comprising: a CDRL1 sequence of SEQ ID NO: 16, a CDRL2 sequence of SEQ ID NO: 20, and a CDRL3 sequence of SEQ ID NO: 24; or (b) a heavy chain variable region comprising: a CDRH1 sequence of SEQ ID NO: 2, a CDRH2 sequence of SEQ ID NO: 8, and a CDRH3 sequence of SEQ ID NO: 13; and a light chain variable region comprising: a CDRL1 sequence of SEQ ID NO: 17, a CDRL2 sequence of SEQ ID NO: 21, and a CDRL3 sequence of SEQ ID NO: 25; or (c) a heavy chain variable region comprising: a CDRH1 sequence of SEQ ID NO: 3, a CDRH2 sequence of SEQ ID NO: 9, and a CDRH3 sequence of SEQ ID NO: 13; and a light chain variable region comprising: a CDRL1 sequence of SEQ ID NO: 17, a CDRL2 sequence of SEQ ID NO: 21, and a CDRL3 sequence of SEQ ID NO: 25; or (d) a heavy chain variable region comprising: a CDRH1 sequence of SEQ ID NO: 4, a CDRH2 sequence of SEQ ID NO: 10, and a CDRH3 sequence of SEQ ID NO: 14; and a light chain variable region comprising: a CDRL1 sequence of SEQ ID NO: 18, a CDRL2 sequence of SEQ ID NO: 22, and a CDRL3 sequence of SEQ ID NO: 24; or (e) a heavy chain variable region comprising: a CDRH1 sequence of SEQ ID NO: 5, a CDRH2 sequence of SEQ ID NO: 11, and a CDRH3 sequence of SEQ ID NO: 15; and a light chain variable region comprising: a CDRL1 sequence of SEQ ID NO: 19, a CDRL2 sequence of SEQ ID NO: 23, and a CDRL3 sequence of SEQ ID NO: 26; or (f) a heavy chain variable region comprising: a CDRH1 sequence of SEQ ID NO: 6, a CDRH2 sequence of SEQ ID NO: 11, and a CDRH3 sequence of SEQ ID NO: 15; and a light chain variable region comprising: a CDRL1 sequence of SEQ ID NO: 19, a CDRL2 sequence of SEQ ID NO: 23, and a CDRL3 sequence of SEQ ID NO: 26; detecting a concentration of the LY6G6D-antibody complex in the biological test sample; and comparing the concentration of the LY6G6D-antibody complex to a reference value to determine whether the subject has or is at risk of developing the disorder.

In some embodiments, the disorder is a cancer. In some embodiments, the cancer is a solid tumor cancer. In some embodiments, the solid tumor cancer is colorectal cancer.

These and further aspects will be further explained in the rest of the disclosure, including the Examples.

### BRIEF DESCRIPTION OF THE DRAWINGS

FIG. 1A-D, are a series of graphs showing binding data from humanized anti-LY6G6D antibodies binding to colon cancer cells expressing different levels of LY6G6D.

FIG. 2A-B, are a series of graphs showing binding data from humanized anti-LY6G6D antibodies binding to CHO-K1 cells expressing human and rhesus LY6G6D.

FIG. 3 is a table summarizing the EC₅₀ values of the cell binding data from FIG. 1 and FIG. 2.

FIG. 4 is a schematic illustration of an LY6G6D×CD3 multispecific antibody in accordance with embodiments of the invention.

FIG. 5A-D, are a series of graphs showing binding data from LY6G6D×CD3 antibodies binding to colon cancer cells expressing LY6G6D and T-cells expressing CD3.

FIG. 6 is a table summarizing the EC₅₀ values of the cell binding data from FIG. 5.

FIG. 7A-H, are a series of graphs showing cytokine release data from LY6G6D×CD3 antibodies binding to colon cancer cells expressing LY6G6D and T-cells expressing CD3.

FIG. 8A-D, are a series of graphs showing T-cell activation data from LY6G6D×CD3 antibodies binding to colon cancer cells expressing LY6G6D and T-cells expressing CD3.

FIG. 9A-D, are a series of graphs showing cytotoxicity data from LY6G6D×CD3 antibodies binding to colon cancer cells expressing LY6G6D and T-cells expressing CD3.

FIG. 10 is a graph showing anti-tumor efficacy of LY6G6D×CD3 bispecific antibodies in accordance with embodiments of the invention, in an HT55 colon cancer cell model in NSG mice using 0.3 to 1 mg/kg biweekly dosing.

FIG. 11 is a graph showing anti-tumor efficacy of LY6G6D×CD3 bispecific antibodies in accordance with embodiments of the invention, in an HT55 colon cancer cell model in NSG mice using 0.3 mg/kg biweekly dosing.

FIG. 12 is a graph showing anti-tumor efficacy of LY6G6D×CD3 bispecific antibodies in accordance with embodiments of the invention, in an HT55 colon cancer cell model in NSG mice using 0.1 mg/kg weekly dosing.

FIG. 13 is a graph showing anti-tumor efficacy of LY6G6D×CD3 bispecific antibodies in accordance with embodiments of the invention, in a COLO32DM colon cancer cell model in NSG mice using weekly intravenous dosing at 0.1 to 1 mg/kg.

FIG. 14 is a graph showing PK data for LY6G6D×CD3 bispecific antibodies in accordance with embodiments of the invention, with (QL) or without (WT) FcRn binding mutations.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, cd., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health. Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

Antibodies, also referred to as immunoglobulins, conventionally comprise at least one heavy chain and one light chain, where the amino terminal domain of the heavy and light chains is variable in sequence, hence is commonly referred to as a variable region domain, or a variable heavy (VH) or variable light (VL) domain. The two domains conventionally associate to form a specific binding region, although as will be discussed here, specific binding can also be obtained with heavy chain-only variable sequences, and a variety of non-natural configurations of antibodies are known and used in the art.

A “functional” or “biologically active” antibody or antigen-binding molecule (including multi-specific (e.g., bispecific) antibodies) is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signal transduction or enzymatic activity. A functional antibody or other binding molecule may also block ligand activation of a receptor or act as an agonist or antagonist. The capability of an antibody or other binding molecule to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), heavy chain-only antibodies, three chain antibodies, TCAs, single chain Fv (scFv), nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule, or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgAQ1 and IgA2) or subclass of immunoglobulin molecule, including engineered subclasses with altered Fc portions that provide for reduced or enhanced effector cell activity. Light chains of the subject antibodies can be kappa light chains (Vkappa) or lambda light chains (Vlambda). The immunoglobulins can be derived from any species. In one aspect, the immunoglobulin is of largely human origin.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site.

Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present invention can be made by the hybridoma method first described by Kohler et al. (1975) Nature 256:495, and can also be made via recombinant protein production methods (see, e.g., U.S. Pat. No. 4,816,567), for example.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). In some embodiments, “CDR” means a complementary determining region of an antibody as defined in Lefranc, M P et al., IMGT, the international ImMunoGeneTics database, Nucleic Acids Res., 27:209-212 (1999). “Framework Region” or “FR” residues are those variable domain residues other than the hypervariable region/CDR residues as herein defined.

Exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010:47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B-cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139, each of which is herein specifically incorporated by reference.

An “intact antibody chain” as used herein is one comprising a full-length variable region and a full-length constant region (Fc). An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as a, d, E, Y, and u, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called K (kappa) and 2 (lambda), based on the amino acid sequences of their constant domains. Antibodies in accordance with embodiments of the invention can comprise kappa light chain sequences or lambda light chain sequences.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIIBI; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563; Hezareh et al., (2001) J. Virology 75:12161; U.S. Pat. No. 5,624,821, the disclosures of which are incorporated herein by reference in their entireties). In some embodiments, a variant Fc sequence can include the following amino acid substitutions: L234A; L235A; and G237A. When these three amino acid substitutions are present in an IgG1 Fc sequence, they can be referred to as GIAAA.

Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour K L. et al., 1999 Eur J Immunol. 29 (8): 2613-24; and Shields R L. et al., 2001. J Biol Chem. 276 (9): 6591-604). The human IgG4 Fc amino acid sequence (UniProtKB No. P01861) is provided herein as SEQ ID NO: 76. Silenced IgG1 is described, for example, in Boesch, A. W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of an antibody can comprise one or more mutations in the hinge region to eliminate disulfide bonding. In yet another embodiment, the hinge region of an Fc can be removed entirely. In still another embodiment, an antibody can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

Further, an Fc variant can be constructed to facilitate heterodimerization of desired heavy chain polypeptide subunits, e.g., using knobs-into-holes, or KiH mutations. In some embodiments, a heavy chain polypeptide can include one or more “hole” mutations, such as, e.g., Y349C, T366S, L368A and/or Y407V, or any combination thereof. In some embodiments, a heavy chain polypeptide can include one or more “knob” mutations, such as, e.g., S354C and/or T366W, or any combination thereof. Antibodies in accordance with embodiments of the invention can utilize any suitable combination of knob and hole residues to facilitate desired heterodimer formation.

Fc variants can also be constructed to facilitate half-life extension, e.g., through enhanced FcRn binding. In some embodiments, a heavy chain polypeptide can include a T250Q mutation for this purpose. In some embodiments, a heavy chain polypeptide can include an M428L mutation for this purpose. In some embodiments, a heavy chain polypeptide can include a T250Q mutation and an M428L mutation for this purpose.

Fc variants can also be constructed to facilitate improved purification procedures, e.g., through enhanced protein A binding. In some embodiments, a heavy chain polypeptide can include an H435R mutation for this purpose. In some embodiments, a heavy chain polypeptide can include a Y436F mutation for this purpose. In some embodiments, a heavy chain polypeptide can include an H435R and a Y436F mutation for this purpose.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, an antibody having an Fc region according to this invention can comprise an antibody with or without K447.

Aspects of the invention include antibodies having multi-specific configurations, which include, without limitation, bispecific, trispecific, etc. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 84) (n=1) and GGGGSGGGGS (SEQ ID NO: 85) (n=2). Additional non-limiting examples of linkers include EPKSCDKTHT (SEQ ID NO: 86) and EPKSSDKTHT (SEQ ID NO: 87), which are derived from the natural hinge region of human IgG1. Other suitable linkers can also be used, and are described, for example, in Chen et al., Adv Drug Deliv Rev. 2013 Oct. 15; 65 (10): 1357-69, the disclosure of which is incorporated herein by reference in its entirety. Additional linker sequences may be described elsewhere herein, and can be incorporated into the subject antibodies in any suitable configuration.

Antibodies (e.g., multispecific antibodies) as described herein can be in the form of a dimer, in which two heavy chains are disulfide bonded or otherwise covalently or non-covalently attached to each other, and can optionally include an asymmetric interface between two or more of the CH domains to facilitate proper pairing between polypeptide chains (commonly referred to as a “knobs-into-holes” interface). Knobs into holes antibody engineering techniques for heavy chain heterodimerization are discussed, for example, in Ridgway et al., Protein Eng. 1996 July; 9 (7): 17-21, and U.S. Pat. No. 8,216,805, the disclosures of which are incorporated by reference herein in their entireties. An Fc region comprising an asymmetric interface can be referred to herein with the abbreviation “KiH”, meaning knobs-into-holes. For example, aspects of the invention include a variant Fc region sequence, such as a GIAAA sequence, that contains an asymmetric interface, and which is referred to herein as “GIAAA KiH”.

The term “LY6G6D” as used herein refers to Lymphocyte Antigen 6 Family Member G6D, which belongs to a cluster of leukocyte antigen-6 (LY6) genes located in the major histocompatibility complex (MHC) class III region on chromosome 6. The term “LY6G6D” includes an LY6G6D protein of any human and non-human animal species, and specifically includes human LY6G6D as well as LY6G6D of non-human mammals.

The term “human LY6G6D” as used herein includes any variants, isoforms and species homologs of human LY6G6D (UniProt A0A1U9X7Z8), regardless of its source or mode of preparation. Thus, “human LY6G6D” includes human LY6G6D naturally expressed by cells and LY6G6D expressed on cells transfected with the human LY6G6D gene.

The terms “anti-LY6G6D antibody,” “LY6G6D antibody,” “LY6G6D-binding antibody” are used herein interchangeably to refer to an antibody as hereinabove defined, immunospecifically binding to LY6G6D, including human LY6G6D, as hereinabove defined.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Antibodies of the invention include multi-specific antibodies. Multi-specific antibodies have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. The term “multi-specific antibody” is used herein in the broadest sense and cover all antibodies with more than one binding specificity. The multi-specific anti-LY6G6D antibodies of the present invention specifically include antibodies immunospecifically binding to two or more non-overlapping epitopes on an LY6G6D protein, such as a human LY6G6D (i.e., bivalent and biparatopic). The multi-specific anti-LY6G6D antibodies of the present invention also specifically include antibodies immunospecifically binding to an epitope on an LY6G6D protein, such as human LY6G6D and to an epitope on a different protein, such as, for example, a CD3 protein, such as human CD3 (i.e., bivalent and biparatopic). The multi-specific anti-LY6G6D antibodies of the present invention also specifically include antibodies immunospecifically binding to two or more non-overlapping or partially overlapping epitopes on an LY6G6D protein, such as a human LY6G6D protein, and to an epitope on a different protein, such as, for example, a CD3 protein, such as human CD3 protein (i.e., trivalent and biparatopic).

Antibodies of the invention include monospecific antibodies, having one binding specificity. Monospecific antibodies specifically include antibodies comprising a single binding specificity, as well as antibodies comprising more than one binding unit having the same binding specificity. The terms “monospecific antibody” is used herein in the broadest sense and covers all antibodies with one binding specificity. The monospecific anti-LY6G6D antibodies of the present invention specifically include antibodies immunospecifically binding to one epitope on an LY6G6D protein, such as a human LY6G6D (monovalent and monospecific). The monospecific anti-LY6G6D antibodies of the present invention also specifically include antibodies having more than one binding unit (e.g., multivalent antibodies) immunospecifically binding to an epitope on an LY6G6D protein, such as human LY6G6D. For example, a monospecific antibody in accordance with embodiments of the invention can include two variable regions, each comprising an antigen-binding domain, wherein each antigen-binding domain binds to the same epitope on an LY6G6D protein (i.e., bivalent and monospecific).

An “epitope” is the site on the surface of an antigen molecule to which a single antibody molecule binds. Generally, an antigen has several or many different epitopes and reacts with many different antibodies. The term specifically includes linear epitopes and conformational epitopes.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

“Polyepitopic specificity” refers to the ability to specifically bind to two or more different epitopes on the same or different target(s). As noted above, the present invention specifically includes anti-LY6G6D antibodies with polyepitopic specificities. i.e., anti-LY6G6D antibodies binding to one or more non-overlapping epitopes on an LY6G6D protein, such as a human LY6G6D; and anti-LY6G6D antibodies binding to one or more epitopes on an LY6G6D protein and to an epitope on a different protein, such as, for example, a CD3 protein. The term “non-overlapping epitope(s)” or “non-competitive epitope(s)” of an antigen is defined herein to mean epitope(s) that are recognized by one member of a pair of antigen-specific antibodies but not the other member. Pairs of antibodies, or antigen-binding regions targeting the same antigen on a multi-specific antibody, recognizing non-overlapping epitopes, do not compete for binding to that antigen and are able to bind that antigen simultaneously.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in all number of different formats, using either labeled antigen or labeled antibody. Usually, the antigen is immobilized on a 96-well plate, and the ability of unlabeled antibodies to block the binding of labeled antibodies is measured using radioactive or enzyme labels.

The term “valent” as used herein refers to a specified number of binding sites in an antibody molecule.

A “monovalent” antibody has one binding site. Thus, a monovalent antibody is also monospecific.

A “multi-valent” antibody has two or more binding sites. Thus, the terms “bivalent”, “trivalent”, and “tetravalent” refer to the presence of two binding sites, three binding sites, and four binding sites, respectively. Thus, a bispecific antibody according to the invention is at least bivalent and may be trivalent, tetravalent, or otherwise multi-valent. A bivalent antibody in accordance with embodiments of the invention may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB), tri-specific antibodies, and the like.

The term “chimeric antigen receptor” or “CAR” is used herein in the broadest sense to refer to an engineered receptor, which grafts a desired binding specificity (e.g., the antigen-binding region of a monoclonal antibody or other ligand) to membrane-spanning and intracellular-signaling domains. Typically, the receptor is used to graft the specificity of a monoclonal antibody onto a T-cell to create a chimeric antigen receptors (CAR). (J Natl Cancer Inst, 2015; 108 (7): dvj439; and Jackson et al., Nature Reviews Clinical Oncology, 2016; 13:370-383). CAR-T cells are T-cells that have been genetically engineered to produce an artificial T-cell receptor for use in immunotherapy. In one embodiment, “CAR-T cell” means a therapeutic T-cell expressing a transgene encoding one or more chimeric antigen receptors comprised minimally of an extracellular domain, a transmembrane domain, and at least one cytosolic domain.

The term “human antibody” is used herein to include antibodies having variable and constant regions derived from human germline immunoglobulin sequences. The human antibodies herein may include amino acid residues not encoded by human germline immunoglobulin sequences. e.g., mutations introduced by random or site-specific mutagenesis in vitro or by somatic mutation in vivo.

By a “chimeric antibody” or a “chimeric immunoglobulin” is meant an immunoglobulin molecule comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the invention that have been engineered to produce such chimeric antibodies.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T-cell receptors or FcRs and perform effector functions. Preferably, the cells express at least FcγRIII and perform ADCC effector function. Examples of human leukocytes which mediate ADCC include natural killer (NK) cells, monocytes, cytotoxic T-cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B-cells and T-cells including cytolytic T-cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity (CDC); Fc receptor binding; antibody-dependent cell-mediated cytotoxicity (ADCC); phagocytosis; down regulation of cell surface receptors (e.g., B-cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al., J. Immunol. Methods 202:163 (1996), may be performed.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12 (8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “cancer”, “tumor”, “cancerous”, and “malignant” as used interchangeably herein refer to or describe the physiological condition in mammals that is typically characterized by unregulated cell growth. Examples of cancers include, but are not limited to, carcinoma including adenocarcinomas, lymphomas, blastomas, melanomas, sarcomas, and leukemias.

The terms “hematologic cancer”, “hematologic malignancy” and “blood cancer” are used interchangeably herein to refer to cancers that begin in blood-forming tissues, including, but not limited to, bone marrow and/or cells of the immune system. Non-limiting examples of hematologic cancers include leukemia, lymphoma, myeloma, or myelodysplastic syndrome. In some embodiments, the leukemia is an acute myeloid leukemia (AML), acute lymphoblastic leukemia (ALL), chronic myelogenous leukemia (CML), or chronic lymphocytic leukemia (CLL). In some embodiments, the lymphoma is Hodgkin's lymphoma or non-Hodgkin's lymphoma.

The term “characterized by expression of LY6G6D” broadly refers to any disease or disorder in which LY6G6D expression is associated with or involved with one or more pathological processes that are characteristic of the disease or disorder. Such disorders include, but are not limited to, colorectal cancer.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10:29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, etc.

### II Detailed Description

### Anti-LY6G6D Antibodies

The present invention provides a family of closely related antibodies that bind to human LY6G6D. The antibodies of this family comprise a set of CDRH sequences as defined herein and shown in Table 1 and a set of CDRL sequences as defined herein and showing in Table 2.

Anti-LY6G6D antibodies in accordance with embodiments of the invention are exemplified by the provided heavy chain variable region (VH) sequences set forth in Table 3, and the provided light chain variable region (VL) sequences set forth in Table 4. The family of antibodies provides a number of benefits that contribute to utility as clinically therapeutic agent(s). The antibodies include members with a range of binding affinities, allowing the selection of a specific sequence with a desired binding affinity.

TABLE 1
Anti-LY6G6D antibody CDRH amino acid sequences.

| Clone ID | HCDR1 | HCDR2 | HCDR3 |
|---|---|---|---|
| 1E3 | GFWMS | DIDSSGSVRNYAPSIKD | AMDY |
| | (SEQ ID | (SEQ ID NO: 7) | (SEQ ID |
| | NO: 1) | | NO: 12) |
| 2G8 | SYSMH | RIDPISGGTRYNEMFKN | FTMLVDH |
| | (SEQ ID | (SEQ ID NO: 8) | (SEQ ID |
| | NO: 2) | | NO: 13) |
| hu2G8 | SYSMH | RIDPISGGTRYNEMFKN | FTMLVDH |
| | (SEQ ID | (SEQ ID NO: 8) | (SEQ ID |
| | NO: 2) | | NO: 13) |
| hu2G8.B6 | SYSIH | RIDPISGGTLYNEMFKN | FTMLVDH |
| | (SEQ ID | (SEQ ID NO: 9) | (SEQ ID |
| | NO: 3) | | NO: 13) |
| 4C10 | DFWMS | DINSDGSAINYAPSLKD | SLDY |
| | (SEQ ID | (SEQ ID NO: 10) | (SEQ ID |
| | NO: 4) | | NO: 14) |
| 7D4 | SYGIT | VVWPSGATNYNSALNS | WDY |
| | (SEQ ID | (SEQ ID NO: 11) | (SEQ ID |
| | NO: 5) | | NO: 15) |
| hu7D4 | SYGIT | VVWPSGATNYNSALNS | WDY |
| | (SEQ ID | (SEQ ID NO: 11) | (SEQ ID |
| | NO: 5) | | NO: 15) |
| hu7D4.B4 | SYGIL | VVWPSGATNYNSALNS | WDY |
| | (SEQ ID | (SEQ ID NO: 11) | (SEQ ID |
| | NO: 6) | | NO: P15) |

TABLE 2
Anti-LY6G6D antibody CDRL amino acid sequences.

| Clone ID | LCDR1 | LCDR2 | LCDR3 |
|---|---|---|---|
| 1E3 | RSSQSIVHRDGNTYLA | KLSYRFS | LQGSHIPLT |
| | (SEQ ID NO: 16) | (SEQ ID | (SEQ ID |
| | | NO: 20) | NO: 24) |
| 2G8 | KSSQSLLYSDGRTYLN | LVSKLDS | WQGTHFPQT |
| | (SEQ ID NO: 17) | (SEQ ID | (SEQ ID |
| | | NO: 21) | NO: 25) |
| hu2G8 | KSSQSLLYSDGRTYLN | LVSKLDS | WQGTHFPQT |
| | (SEQ ID NO: 17) | (SEQ ID | (SEQ ID |
| | | NO: 21) | NO: 25) |
| hu2G8.B6 | KSSQSLLYSDGRTYLN | LVSKLDS | WQGTHFPQT |
| | (SEQ ID NO: 17) | (SEQ ID | (SEQ ID |
| | | NO: 21) | NO: 25) |
| 4C10 | RSSQSIVHSNGNTFLA | KVSNRFS | LQGSHIPLT |
| | (SEQ ID NO: 18) | (SEQ ID | (SEQ ID |
| | | NO: 22) | NO: 24) |
| 7D4 | RSSKSLLHSNGNTYLY | RVSNLAS | MQHLEYPYT |
| | (SEQ ID NO: 19) | (SEQ ID | (SEQ ID |
| | | NO: 23) | NO: 26) |
| hu7D4 | RSSKSLLHSNGNTYLY | RVSNLAS | MQHLEYPYT |
| | (SEQ ID NO: 19) | (SEQ ID | (SEQ ID |
| | | NO: 23) | NO: 26) |
| hu7D4.B4 | RSSKSLLHSNGNTYLY | RVSNLAS | MQHLEYPYT |
| | (SEQ ID NO: 19) | (SEQ ID | (SEQ ID |
| | | NO: 23) | NO: 26) |

TABLE 3
Anti-LY6G6D heavy chain variable
domain amino acid sequences.

| Clone ID | Heavy chain variable region sequence |
|---|---|
| 1E3 | EVQLLETGGGLVQPGGSRRLSCEGSGFTFSGFWMSWVRQ |
| | TPGKTLEWIGDIDSSGSVRNYAPSIKDRFTFFRDNDKNT |
| | LYLQMSNVRSEDTATYFCLRAMDYWGHGTSVTVSS |
| | (SEQ ID NO: 27) |
| 2G8 | QVQLQQPGAELVKPGTSVKLSCKASGYTFTSYSMHWVKQ |
| | RPGRGLEWIGRIDPISGGTRYNEMFKNKATLTVDVPSST |
| | AYMQLNSLTSEDSAVYYCARFTMLVDHWGQGTTLTVSS |
| | (SEQ ID NO: 28) |
| hu2G8 | EVQLVQSGAEVKKPGASVKVSCKASGYTFTSYSMHWVRQ |
| | APGQGLEWIGRIDPISGGTRYNEMFKNWATLTVDVSIST |
| | AYMELSRLRSDDTAVYYCARFTMLVDHWGQGTTVTVSS |
| | (SEQ ID NO: 29) |
| hu2G8.B6 | EVQLVQSGAEVKKPGASVKVSCKASGYTFTSYSIHWVRQ |
| | APGQGLEWIGRIDPISGGTLYNEMFKNWATLTVDVSIST |
| | AYMELSRLRSDDTAVYYCARFTMLVDHWGQGTTVTVSS |
| | (SEQ ID NO: 30) |
| 4C10 | EVQLLETGGGPVQPGGSRGLSCEGSGFTFSDFWMSWVRQ |
| | TPGKTLEWIGDINSDGSAINYAPSLKDRFTIFRDNDKST |
| | LHLQMSNVRSEDTATYFCVKSLDYWGQGISVTVSS |
| | (SEQ ID NO: 31) |
| 7D4 | QVQLKESGPGLVAPSQSLSITCTVSGFSLTSYGITWVRQ |
| | PPGKGLEWLGVVWPSGATNYNSALNSRLSISKDNSKSQV |
| | FLKMNSLQTDDTARYYCARWDYWGQGTTLTVSS |
| | (SEQ ID NO: 32) |
| hu7D4 | EVQLVQSGAEVKKPGASVKVSCKVSGFSLTSYGITWVRQ |
| | APGQGLEWLGVVWPSGATNYNSALNSRLTITKDNSTSTV |
| | YMELRSLRSDDTAVYYCARWDYWGQGTTVTVSS |
| | (SEQ ID NO: 33) |
| hu7D4.B4 | EVQLVQSGAEVKKPGASVKVSCKVSGFSLTSYGILWVRQ |
| | APGQGLEWLGVVWPSGATNYNSALNSRLTITKDTSTSTV |
| | YMELRSLRSDDTAVYYCARWDYWGQGTTVTVSS |
| | (SEQ ID NO: 34) |

TABLE 4
Anti-LY6G6D light chain variable
domain amino acid sequences.

| Clone ID | Light chain variable region sequence |
|---|---|
| 1E3 | EVLMTQTPLSLPVSLGDQASISCRSSQSIVHRDGNTYLA |
| | WYLQKPGQSPKLLIYKLSYRFSGVPDRFSGSGSGTDFTL |
| | RISRVEAEDLGLYYCLQGSHIPLTFGAGTKLELK |
| | (SEQ ID NO: 35) |
| 2G8 | DVVMTQTPLTLSVTIGQPASISCKSSQSLLYSDGRTYLN |
| | WMLQRPGQSPKRLIYLVSKLDSGVPDRFTGSGSGTDFTL |
| | KISRVEAEDLGIYYCWQGTHFPQTFGGGTKLEIK |
| | (SEQ ID NO: 36) |
| hu2G8 | DVVMTQSPLSLPVTLGQPASISCKSSQSLLYSDGRTYLN |
| | WMQQRPGQSPRRLIYLVSKLDSGVPDRFSGSGSGTDFTL |
| | KISRVEAEDVGVYYCWQGTHFPQTFGGGTKVEIK |
| | (SEQ ID NO: 37) |
| hu2G8.B6 | DVVMTQSPLSLPVTLGQPASISCKSSQSLLYSDGRTYLN |
| | WMQQRPGQSPRRLIYLVSKLDSGVPDRFSGSGSGTDFTL |
| | KISRVEAEDVGVYYCWQGTHFPQTFGGGTKVEIK |
| | (SEQ ID NO: 38) |
| 4C10 | DILMTQNPLSLSVSLGDQASISCRSSQSIVHSNGNTFLA |
| | WYLQKPGQSPKLLIYKVSNRFSGVPDRFSGSGSGTDFTL |
| | RISRVEAEDLGIYYCLQGSHIPLTFGAGTTLELK |
| | (SEQ ID NO: 39) |
| 7D4 | DIVMTQAAPSVPVTPGESVSISCRSSKSLLHSNGNTYLY |
| | WFLQRPGQSPQLLIYRVSNLASGVPDRFGGSGSGTAFTL |
| | RISRVEAEDVGVYYCMQHLEYPYTFGGGTKLEIK |
| | (SEQ ID NO: 40) |
| hu7D4 | DIVMTQSPLSLPVTPGEPASISCRSSKSLLHSNGNTYLY |
| | WFLQKPGQSPQLLIYRVSNLASGVPDRFSGSGSGTDFTL |
| | KISRVEAEDVGVYYCMQHLEYPYTFGQGTKLEIK |
| | (SEQ ID NO: 41) |
| hu7D4.B4 | DIVMTQSPLSLPVTPGEPASISCRSSKSLLHSNGNTYLY |
| | WFLQKPGQSPQLLIYRVSNLASGVPDRFSGSGSGTDFTL |
| | KISRVEAEDVGVYYCMQHLEYPYTFGQGTKLEIK |
| | (SEQ ID NO: 42) |

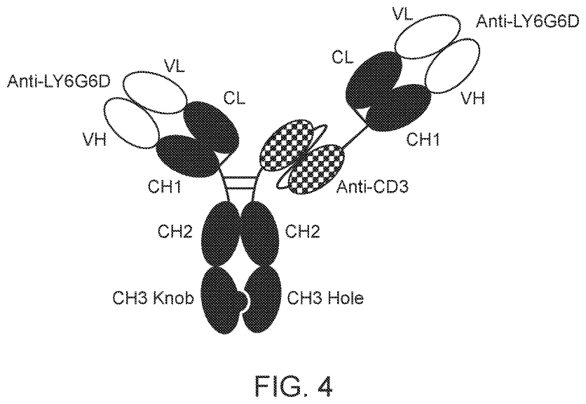

A suitable antibody may be selected from those provided herein for development and therapeutic or other use, including, without limitation, use as a bispecific antibody, e.g., as shown in FIG. 4, or part of a CAR-T structure. FIG. 4 is a schematic illustration of anti-LY6G6D×anti-CD38 multi-specific antibody, where the anti-LY6G6D binding regions are located at the N-termini of the polypeptide subunits, and the CD3& binding unit is in an scFv format, and is located on one of the heavy chain polypeptide subunits between the variable region and the hinge region. In some embodiments, the two heavy chain polypeptide subunits of a heterodimeric multispecific antibody are pared using, e.g., knobs-into-holes (KiH) technology.

Determination of affinity for a candidate protein can be performed using methods known in the art, such as Biacore measurements. Members of the antibody family may have an affinity for LY6G6D with a Kd of from about 10⁻⁶ to around about 10⁻¹¹, including without limitation: from about 10⁻⁶ to around about 10⁻¹⁰; from about 10⁻⁶ to around about 10⁻⁹; from about 106 to around about 10⁻⁸: from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating, e.g., blocking, an LY6G6D biological activity, including in vitro assays, pre-clinical models, and clinical trials, as well as assessment of potential toxicity.

Members of the antibody family herein are cross-reactive with the LY6G6D protein of Cynomolgus macaque, and can be engineered to provide cross-reactivity with the LY6G6D protein of any other animal species, if desired.

The family of anti-LY6G6D antibodies herein comprises one or more binding units comprising a VH domain, comprising CDRH1, CDRH2 and CDRH3 sequences in a human VH framework, and CDRL1, CDRL2 and CDRL3 sequences in a human Vkappa or Vlambda framework. The CDR sequences may be situated, as an example, in the region of around amino acid residues 26-33; 51-58; and 97-116 for CDR1, CDR2 and CDR3, respectively, of the provided exemplary variable region sequences set forth in Tables 3 and 4. It will be understood by one of ordinary skill in the art that the CDR sequences may be in different positions if a different framework sequence is selected, although generally the order of the sequences will remain the same.

In some embodiments, an anti-LY6G6D antibody comprises a CDRH1 sequence of any one of SEQ ID NOs: 1-6. In some embodiments, an anti-LY6G6D antibody comprises a CDRH2 sequence of any one of SEQ ID NOs: 7-11. In some embodiments, an anti-LY6G6D antibody comprises a CDRH3 sequence of any one of SEQ ID NOs: 12-15.

In one preferred embodiment, an anti-LY6G6D antibody comprises a CDRH1 sequence of SEQ ID NO: 1, a CDRH2 sequence of SEQ ID NO: 7, and a CDRH3 sequence of SEQ ID NO: 12. In one preferred embodiment, an anti-LY6G6D antibody comprises a CDRH1 sequence of SEQ ID NO: 2, a CDRH2 sequence of SEQ ID NO: 8, and a CDRH3 sequence of SEQ ID NO: 13. In one preferred embodiment, an anti-LY6G6D antibody comprises a CDRH1 sequence of SEQ ID NO: 2, a CDRH2 sequence of SEQ ID NO: 8, and a CDRH3 sequence of SEQ ID NO: 13. In one preferred embodiment, an anti-LY6G6D antibody comprises a CDRH1 sequence of SEQ ID NO: 3, a CDRH2 sequence of SEQ ID NO: 9, and a CDRH3 sequence of SEQ ID NO: 13. In one preferred embodiment, an anti-LY6G6D antibody comprises a CDRH1 sequence of SEQ ID NO: 4, a CDRH2 sequence of SEQ ID NO: 10, and a CDRH3 sequence of SEQ ID NO: 14. In one preferred embodiment, an anti-LY6G6D antibody comprises a CDRH1 sequence of SEQ ID NO: 5, a CDRH2 sequence of SEQ ID NO: 11, and a CDRH3 sequence of SEQ ID NO: 15. In one preferred embodiment, an anti-LY6G6D antibody comprises a CDRH1 sequence of SEQ ID NO: 6, a CDRH2 sequence of SEQ ID NO: 11, and a CDRH3 sequence of SEQ ID NO: 15.

In some embodiments, an anti-LY6G6D antibody comprises a CDRL1 sequence of any one of SEQ ID NOs: 16-19. In some embodiments, an anti-LY6G6D antibody comprises a CDRL2 sequence of any one of SEQ ID NOs: 20-23. In some embodiments, an anti-LY6G6D antibody comprises a CDRL3 sequence of any one of SEQ ID NOs: 24-26.

In one preferred embodiment, an anti-LY6G6D antibody comprises a CDRL1 sequence of SEQ ID NO: 16, a CDRL2 sequence of SEQ ID NO: 20, and a CDRL3 sequence of SEQ ID NO: 24. In one preferred embodiment, an anti-LY6G6D antibody comprises a CDRL1 sequence of SEQ ID NO: 17, a CDRL2 sequence of SEQ ID NO: 21, and a CDRL3 sequence of SEQ ID NO: 25. In one preferred embodiment, an anti-LY6G6D antibody comprises a CDRL1 sequence of SEQ ID NO: 18, a CDRL2 sequence of SEQ ID NO: 22, and a CDRL3 sequence of SEQ ID NO: 24. In one preferred embodiment, an anti-LY6G6D antibody comprises a CDRL1 sequence of SEQ ID NO: 19, a CDRL2 sequence of SEQ ID NO: 23, and a CDRL3 sequence of SEQ ID NO: 26.

In one preferred embodiment, an anti-LY6G6D antibody comprises a heavy chain variable region comprising: a CDRH1 sequence of SEQ ID NO: 1, a CDRH2 sequence of SEQ ID NO: 7, and a CDRH3 sequence of SEQ ID NO: 12; and a light chain variable region comprising: a CDRL1 sequence of SEQ ID NO: 16, a CDRL2 sequence of SEQ ID NO: 20, and a CDRL3 sequence of SEQ ID NO: 24.

In one preferred embodiment, an anti-LY6G6D antibody comprises a heavy chain variable region comprising: a CDRH1 sequence of SEQ ID NO: 2, a CDRH2 sequence of SEQ ID NO: 8, and a CDRH3 sequence of SEQ ID NO: 13; and a light chain variable region comprising: a CDRL1 sequence of SEQ ID NO: 17, a CDRL2 sequence of SEQ ID NO: 21, and a CDRL3 sequence of SEQ ID NO: 25.

In one preferred embodiment, an anti-LY6G6D antibody comprises a heavy chain variable region comprising: a CDRH1 sequence of SEQ ID NO: 3, a CDRH2 sequence of SEQ ID NO: 9, and a CDRH3 sequence of SEQ ID NO: 13; and a light chain variable region comprising: a CDRL1 sequence of SEQ ID NO: 17, a CDRL2 sequence of SEQ ID NO: 21, and a CDRL3 sequence of SEQ ID NO: 25.

In one preferred embodiment, an anti-LY6G6D antibody comprises a heavy chain variable region comprising: a CDRH1 sequence of SEQ ID NO: 4, a CDRH2 sequence of SEQ ID NO: 10, and a CDRH3 sequence of SEQ ID NO: 14; and a light chain variable region comprising: a CDRL1 sequence of SEQ ID NO: 18, a CDRL2 sequence of SEQ ID NO: 22, and a CDRL3 sequence of SEQ ID NO: 24.

In one preferred embodiment, an anti-LY6G6D antibody comprises a heavy chain variable region comprising: a CDRH1 sequence of SEQ ID NO: 5, a CDRH2 sequence of SEQ ID NO: 11, and a CDRH3 sequence of SEQ ID NO: 15; and a light chain variable region comprising: a CDRL1 sequence of SEQ ID NO: 19, a CDRL2 sequence of SEQ ID NO: 23, and a CDRL3 sequence of SEQ ID NO: 26.

In one preferred embodiment, an anti-LY6G6D antibody comprises a heavy chain variable region comprising: a CDRH1 sequence of SEQ ID NO: 6, a CDRH2 sequence of SEQ ID NO: 11, and a CDRH3 sequence of SEQ ID NO: 15; and a light chain variable region comprising: a CDRL1 sequence of SEQ ID NO: 19, a CDRL2 sequence of SEQ ID NO: 23, and a CDRL3 sequence of SEQ ID NO: 26.

In some embodiments, an anti-LY6G6D antibody comprises any of the heavy chain variable region amino acid sequences of SEQ ID NOs: 27-34 (Table 3). In some embodiments, an anti-LY6G6D antibody comprises a heavy chain variable region amino acid sequence that has at least about 80% identity, such as about 85%, 90%, 95%, 99% or 100% identity to the sequence of any one of SEQ ID NOs: 27-34 (Table 3).

In some embodiments, an anti-LY6G6D antibody comprises any of the light chain variable region amino acid sequences of SEQ ID NOs: 35-42 (Table 4). In some embodiments, an anti-LY6G6D antibody comprises a light chain variable region amino acid sequence that has at least about 80% identity, such as about 85%, 90%, 95%, 99% or 100% identity to the sequence of any one of SEQ ID NOs: 35-42 (Table 4).

In some embodiments, an anti-LY6G6D antibody comprises a heavy chain variable region sequence of SEQ ID NO: 27 and a light chain variable region sequence of SEQ ID NO: 35. In some embodiments, an anti-LY6G6D antibody comprises a heavy chain variable region sequence of SEQ ID NO: 28 and a light chain variable region sequence of SEQ ID NO: 36. In some embodiments, an anti-LY6G6D antibody comprises a heavy chain variable region sequence of SEQ ID NO: 29 and a light chain variable region sequence of SEQ ID NO: 37. In some embodiments, an anti-LY6G6D antibody comprises a heavy chain variable region sequence of SEQ ID NO: 30 and a light chain variable region sequence of SEQ ID NO: 38. In some embodiments, an anti-LY6G6D antibody comprises a heavy chain variable region sequence of SEQ ID NO: 31 and a light chain variable region sequence of SEQ ID NO: 39. In some embodiments, an anti-LY6G6D antibody comprises a heavy chain variable region sequence of SEQ ID NO: 32 and a light chain variable region sequence of SEQ ID NO: 40. In some embodiments, an anti-LY6G6D antibody comprises a heavy chain variable region sequence of SEQ ID NO: 33 and a light chain variable region sequence of SEQ ID NO: 41. In some embodiments, an anti-LY6G6D antibody comprises a heavy chain variable region sequence of SEQ ID NO: 34 and a light chain variable region sequence of SEQ ID NO: 42.

In some embodiments, a CDR sequence in an anti-LY6G6D antibody of the invention comprises one or two amino acid substitutions relative to a CDR1, CDR2 and/or CDR3 sequence or set of CDR1, CDR2 and CDR3 sequences in Table 1 or 2.

In some embodiments, an anti-LY6G6D antibody preferably comprises a heavy chain variable domain (VH) in which the CDRH3 sequence has greater than or equal to 80%, such as at least 85%, at least 90%, at least 95%, or at least 99% sequence identity at the amino acid level to a CDRH3 sequence of any one of the antibodies whose CDRH3 sequences are provided in Table 1, and binds to LY6G6D.

In some embodiments, an anti-LY6G6D antibody preferably comprises a heavy chain variable domain (VH) in which the full set of CDRHs 1, 2, and 3 (combined) has greater than or equal to eighty-five percent (85%) sequence identity at the amino acid level to the CDRHs 1, 2, and 3 (combined) of the antibodies whose CDRH sequences are provided in Table 1, and binds to LY6G6D.

In some embodiments, bispecific or multi-specific antibodies are provided, which may have any of the configurations discussed herein, including, without limitation, multispecific antibodies comprising one or more binding units located on a heavy chain polypeptide subunit, and placed in between an N-terminal binding unit and the hinge region, as shown, for example, in FIG. 4. In some embodiments, such a binding unit can comprise an scFv configuration, wherein a VH and VL region are connected by a linker sequence, and together form a binding unit that binds to a target protein, such, for example, CD3.

In some embodiments, a multi-specific antibody can comprise a first binding unit that binds to LY6G6D, comprising a heavy chain variable region paired with a light chain variable region, and a second binding unit that binds to a protein other than LY6G6D, comprising a heavy chain variable region paired with a light chain variable region. In some embodiments, a multispecific antibody can comprise a first and second binding unit, each of which comprise a heavy chain variable region paired with a light chain variable region, and both of which bind to LY6G6D, and at least a third binding unit that binds to a protein other than LY6G6D, such as, for example, CD3. In some embodiments, the third binding unit is positioned at a C-terminus of one of the light chain polypeptide subunits that make up the antibody. In some embodiments, the third binding unit is positioned within one of the heavy chain polypeptide subunits, e.g., between the variable region and the hinge region of the polypeptide subunit, as depicted, for example, in FIG. 4. In some embodiments, the third binding unit comprises an scFv, wherein a heavy chain variable region is connected to a light chain variable region with a linker sequence.

In some embodiments, the anti-LY6G6D antibodies described herein comprise heavy chain constant region sequences, such as CH1, hinge, CH2, CH3 and/or CH4 domains. In some embodiments, the anti-LY6G6D antibodies described herein comprise light chain constant region sequences, such as CL domains. In some embodiments, the anti-LY6G6D antibodies described herein comprise heavy chain constant region sequences that form an Fc region.

As noted above, in some embodiments, a multi-specific anti-LY6G6D antibody comprises a binding unit that binds to a protein other than LY6G6D, such as, for example, CD3 (e.g., CD38). In some embodiments, a CD3 binding unit comprises a set of CDRH sequences as defined herein and shown in Table 5, and a set of CDRL sequences as defined herein and showing in Table 6.

TABLE 5
CD3 heavy chain CDR sequences:

| Clone ID | HCDR1 | HCDR2 | HCDR3 |
|---|---|---|---|
| 1B4 | TYAMN | RIRSKYNNYATYYADSVKD | HGNFGNSYV |
| | (SEQ ID | | SWFAY |
| | NO: 43) | (SEQ ID NO: 44) | (SEQ ID |
| | | | NO: 46) |
| 1B10 | TYAMN | RIRSKANNYATYYADSVKG | HGNFGNSYV |
| | (SEQ ID | | SWFAY |
| | NO: 43) | (SEQ ID NO: 45) | (SEQ ID |
| | | | NO: 46) |

TABLE 6
CD3 light chain CDR sequences:

| Clone ID | LCDR1 | LCDR2 | LCDR3 |
|---|---|---|---|
| 1B4 | GSSTGAVTTSNYAN | GTNKRAP | ALWYSNLWV |
| | (SEQ ID NO: 47) | (SEQ ID | (SEQ ID NO: 49) |
| | | NO: 48) | |
| 1B10 | GSSTGAVTTSNYAN | GTNKRAP | ALWYSNLWV |
| | (SEQ ID NO: 47) | (SEQ ID | (SEQ ID NO: 49) |
| | | NO: 48) | |

In some embodiments, a CD3 binding unit comprises a heavy chain variable region (VH) sequence set forth in Table 7, and a light chain variable region (VL) sequence set forth in Table 8. These CD3 binding units provide a number of benefits that contribute to utility as clinically therapeutic agent(s).

TABLE 7
CD3 heavy chain variable region sequences

| Clone ID | Heavy chain variable region sequence |
|---|---|
| 1B4 | EVKLVESGGGLVQPGGSLKLSCAASGFTFNTYAMNWVRQ |
| | APGKGLEWVGRIRSKYNNYATYYADSVKDRFTISRDDSQ |
| | NILYLQMNNLRTEDTAVYYCVRHGNFGNSYVSWFAYWGQ |
| | GTLVTVSS |
| | (SEQ ID NO: 50) |
| 1B10 | EVKLLESGGGLVQPGGSLKLSCAASGFTFNTYAMNWVRQ |
| | APGKGLEWVGRIRSKANNYATYYADSVKGRFTISRDDSK |
| | NTLYLQMNNLKAEDTAVYYCVRHGNFGNSYVSWFAYWGQ |
| | GTLVTVSS |
| | (SEQ ID NO: 51) |

TABLE 8
CD3 light chain variable region sequences

| Clone ID | Light chain variable region sequence |
|---|---|
| 1B4 | QAFVTQEPSLTVSPGGTVTLTCGSSTGAVTTSNYANWVQ |
| | QKPGQAPRGLIGGTNKRAPGVPARFSGSLIGDKAALTIT |
| | GAQPEDEAEYYCALWYSNLWVFGGGTKLTVL |
| | (SEQ ID NO: 52) |
| 1B10 | EAVVTQEPSLTVSPGGTVTLTCGSSTGAVTTSNYANWVQ |
| | QKPGQAPRGLIGGTNKRAPGTPARFSGSLLGDKAALTIT |
| | GAQPEDEAEYYCALWYSNLWVFGGGTKLTVL |
| | (SEQ ID NO: 53) |

Multi-specific anti-LY6G6D antibodies in accordance with embodiments of the invention can include one or more CD3 binding units, as described herein. Any of the binding domains of the anti-LY6G6D antibodies described herein can be combined with the CD3-binding domains described herein to generate multi-specific antibodies that bind to LY6G6D and CD3. Full length sequences that can be used to compose an anti-LY6G6D×CD3 antibody are provided in Table 9.

TABLE 9
LY6G6D x CD3 antibody full length polypeptide subunit sequences

| Name | Sequence |
|---|---|
| hu2G8_1B10_G1AAA_hole_RF | EVQLVQSGAEVKKPGASVKVSCKASGYTFTSYSMHW |
| | VRQAPGQGLEWIGRIDPISGGTRYNEMFKNWATLTVD |
| | VSISTAYMELSRLRSDDTAVYYCARFTMLVDHWGQG |
| | TTVTVSSASTKGPSVFPLAPSSKSTSGGTAALGCLVKD |
| | YFPEPVTVSWNSGALTSGVHTFPAVLQSSGLYSLSSVV |
| | TVPSSSLGTQTYICNVNHKPSNTKVDKKVEPKSCGGG |
| | GSEVKLLESGGGLVQPGGSLKLSCAASGFTFNTYAMN |
| | WVRQAPGKGLEWVGRIRSKANNYATYYADSVKGRFT |
| | ISRDDSKNTLYLQMNNLKAEDTAVYYCVRHGNFGNS |
| | YVSWFAYWGQGTLVTVSSGGGGSGGGGSGGGGSAE |
| | AVVTQEPSLTVSPGGTVTLTCGSSTGAVTTSNYANWV |
| | QQKPGQAPRGLIGGTNKRAPGTPARFSGSLLGDKAAL |
| | TITGAQPEDEAEYYCALWYSNLWVFGGGTKLTVLGG |
| | GGSDKTHTCPPCPAPEAAGAPSVFLFPPKPKDTLMISR |
| | TPEVTCVVVDVSHEDPEVKFNWYVDGVEVHNAKTKP |
| | REEQYNSTYRVVSVLTVLHQDWLNGKEYKCKVSNKA |
| | LPAPIEKTISKAKGQPREPQVCTLPPSRDELTKNQVSLS |
| | CAVKGFYPSDIAVEWESNGQPENNYKTTPPVLDSDGS |
| | FFLVSKLTVDKSRWQQGNVFSCSVMHEALHNRFTQK |
| | SLSLSPG (SEQ ID NO: 54) |
| hu2G8_1B4_G1AAA_hole_RF | EVQLVQSGAEVKKPGASVKVSCKASGYTFTSYSMHW |
| | VRQAPGQGLEWIGRIDPISGGTRYNEMFKNWATLTVD |
| | VSISTAYMELSRLRSDDTAVYYCARFTMLVDHWGQG |
| | TTVTVSSASTKGPSVFPLAPSSKSTSGGTAALGCLVKD |
| | YFPEPVTVSWNSGALTSGVHTFPAVLQSSGLYSLSSVV |
| | TVPSSSLGTQTYICNVNHKPSNTKVDKKVEPKSCGGG |
| | GSEVKLVESGGGLVQPGGSLKLSCAASGFTFNTYAMN |
| | WVRQAPGKGLEWVGRIRSKYNNYATYYADSVKDRFT |
| | ISRDDSQNILYLQMNNLRTEDTAVYYCVRHGNFGNSY |
| | VSWFAYWGQGTLVTVSSGGGGSGGGGSGGGGSAQA |
| | FVTQEPSLTVSPGGTVTLTCGSSTGAVTTSNYANWVQ |
| | QKPGQAPRGLIGGTNKRAPGVPARFSGSLIGDKAALTI |
| | TGAQPEDEAEYYCALWYSNLWVFGGGTKLTVLGGG |
| | GSDKTHTCPPCPAPEAAGAPSVFLFPPKPKDTLMISRTP |
| | EVTCVVVDVSHEDPEVKFNWYVDGVEVHNAKTKPRE |
| | EQYNSTYRVVSVLTVLHQDWLNGKEYKCKVSNKALP |
| | APIEKTISKAKGQPREPQVCTLPPSRDELTKNQVSLSCA |
| | VKGFYPSDIAVEWESNGQPENNYKTTPPVLDSDGSFFL |
| | VSKLTVDKSRWQQGNVFSCSVMHEALHNRFTQKSLS |
| | LSPG (SEQ ID NO: 55) |
| hu2G8_G1AAA_knob | EVQLVQSGAEVKKPGASVKVSCKASGYTFTSYSMHW |
| | VRQAPGQGLEWIGRIDPISGGTRYNEMFKNWATLTVD |
| | VSISTAYMELSRLRSDDTAVYYCARFTMLVDHWGQG |
| | TTVTVSSASTKGPSVFPLAPSSKSTSGGTAALGCLVKD |
| | YFPEPVTVSWNSGALTSGVHTFPAVLQSSGLYSLSSVV |
| | TVPSSSLGTQTYICNVNHKPSNTKVDKKVEPKSCDKT |
| | HTCPPCPAPEAAGAPSVFLFPPKPKDTLMISRTPEVTCV |
| | VVDVSHEDPEVKFNWYVDGVEVHNAKTKPREEQYNS |
| | TYRVVSVLTVLHQDWLNGKEYKCKVSNKALPAPIEK |
| | TISKAKGQPREPQVYTLPPCRDELTKNQVSLWCLVKG |
| | FYPSDIAVEWESNGQPENNYKTTPPVLDSDGSFFLYSK |
| | LTVDKSRWQQGNVFSCSVMHEALHNHYTQKSLSLSP |
| | G (SEQ ID NO: 56) |
| hu2G8_LC | DVVMTQSPLSLPVTLGQPASISCKSSQSLLYSDGRTYL |
| | NWMQQRPGQSPRRLIYLVSKLDSGVPDRFSGSGSGTD |
| | FTLKISRVEAEDVGVYYCWQGTHFPQTFGGGTKVEIK |
| | RTVAAPSVFIFPPSDEQLKSGTASVVCLLNNFYPREAK |
| | VQWKVDNALQSGNSQESVTEQDSKDSTYSLSSTLTLS |
| | KADYEKHKVYACEVTHQGLSSPVTKSFNRGEC |
| | (SEQ ID NO: 57) |
| hu2G8.B6_1B10_G1AAA_hole_RF | EVQLVQSGAEVKKPGASVKVSCKASGYTFTSYSIHWV |
| | RQAPGQGLEWIGRIDPISGGTLYNEMFKNWATLTVDV |
| | SISTAYMELSRLRSDDTAVYYCARFTMLVDHWGQGT |
| | TVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCGGGG |
| | SEVKLLESGGGLVQPGGSLKLSCAASGFTFNTYAMNW |
| | VRQAPGKGLEWVGRIRSKANNYATYYADSVKGRFTIS |
| | RDDSKNTLYLQMNNLKAEDTAVYYCVRHGNFGNSY |
| | VSWFAYWGQGTLVTVSSGGGGSGGGGSGGGGSAEA |
| | VVTQEPSLTVSPGGTVTLTCGSSTGAVTTSNYANWVQ |
| | QKPGQAPRGLIGGTNKRAPGTPARFSGSLLGDKAALTI |
| | TGAQPEDEAEYYCALWYSNLWVFGGGTKLTVLGGG |
| | GSDKTHTCPPCPAPEAAGAPSVFLFPPKPKDTLMISRTP |
| | EVTCVVVDVSHEDPEVKFNWYVDGVEVHNAKTKPRE |
| | EQYNSTYRVVSVLTVLHQDWLNGKEYKCKVSNKALP |
| | APIEKTISKAKGQPREPQVCTLPPSRDELTKNQVSLSCA |
| | VKGFYPSDIAVEWESNGQPENNYKTTPPVLDSDGSFFL |
| | VSKLTVDKSRWQQGNVFSCSVMHEALHNRFTQKSLS |
| | LSPG (SEQ ID NO: 58) |
| hu2G8.B6_1B4_G1AAA_hole_RF | EVQLVQSGAEVKKPGASVKVSCKASGYTFTSYSIHWV |
| | RQAPGQGLEWIGRIDPISGGTLYNEMFKNWATLTVDV |
| | SISTAYMELSRLRSDDTAVYYCARFTMLVDHWGQGT |
| | TVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCGGGG |
| | SEVKLVESGGGLVQPGGSLKLSCAASGFTFNTYAMN |
| | WVRQAPGKGLEWVGRIRSKYNNYATYYADSVKDRFT |
| | ISRDDSQNILYLQMNNLRTEDTAVYYCVRHGNFGNSY |
| | VSWFAYWGQGTLVTVSSGGGGSGGGGSGGGGSAQA |
| | FVTQEPSLTVSPGGTVTLTCGSSTGAVTTSNYANWVQ |
| | QKPGQAPRGLIGGTNKRAPGVPARFSGSLIGDKAALTI |
| | TGAQPEDEAEYYCALWYSNLWVFGGGTKLTVLGGG |
| | GSDKTHTCPPCPAPEAAGAPSVFLFPPKPKDTLMISRTP |
| | EVTCVVVDVSHEDPEVKFNWYVDGVEVHNAKTKPRE |
| | EQYNSTYRVVSVLTVLHQDWLNGKEYKCKVSNKALP |
| | APIEKTISKAKGQPREPQVCTLPPSRDELTKNQVSLSCA |
| | VKGFYPSDIAVEWESNGQPENNYKTTPPVLDSDGSFFL |
| | VSKLTVDKSRWQQGNVFSCSVMHEALHNRFTQKSLS |
| | LSPG (SEQ ID NO: 59) |
| hu2G8.B6_G1AAA_knob | EVQLVQSGAEVKKPGASVKVSCKASGYTFTSYSIHWV |
| | RQAPGQGLEWIGRIDPISGGTLYNEMFKNWATLTVDV |
| | SISTAYMELSRLRSDDTAVYYCARFTMLVDHWGQGT |
| | TVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCDKTH |
| | TCPPCPAPEAAGAPSVFLFPPKPKDTLMISRTPEVTCVV |
| | VDVSHEDPEVKFNWYVDGVEVHNAKTKPREEQYNST |
| | YRVVSVLTVLHQDWLNGKEYKCKVSNKALPAPIEKTI |
| | SKAKGQPREPQVYTLPPCRDELTKNQVSLWCLVKGFY |
| | PSDIAVEWESNGQPENNYKTTPPVLDSDGSFFLYSKLT |
| | VDKSRWQQGNVFSCSVMHEALHNHYTQKSLSLSPG |
| | (SEQ ID NO: 60) |
| hu2G8.B6_1B4_G1AAA_hole_RF_QL | EVQLVQSGAEVKKPGASVKVSCKASGYTFTSYSIHWV |
| | RQAPGQGLEWIGRIDPISGGTLYNEMFKNWATLTVDV |
| | SISTAYMELSRLRSDDTAVYYCARFTMLVDHWGQGT |
| | TVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCGGGG |
| | SEVKLVESGGGLVQPGGSLKLSCAASGFTFNTYAMN |
| | WVRQAPGKGLEWVGRIRSKYNNYATYYADSVKDRFT |
| | ISRDDSQNILYLQMNNLRTEDTAVYYCVRHGNFGNSY |
| | VSWFAYWGQGTLVTVSSGGGGSGGGGSGGGGSAQA |
| | FVTQEPSLTVSPGGTVTLTCGSSTGAVTTSNYANWVQ |
| | QKPGQAPRGLIGGTNKRAPGVPARFSGSLIGDKAALTI |
| | TGAQPEDEAEYYCALWYSNLWVFGGGTKLTVLGGG |
| | GSDKTHTCPPCPAPEAAGAPSVFLFPPKPKDQLMISRT |
| | PEVTCVVVDVSHEDPEVKFNWYVDGVEVHNAKTKPR |
| | EEQYNSTYRVVSVLTVLHQDWLNGKEYKCKVSNKAL |
| | PAPIEKTISKAKGQPREPQVCTLPPSRDELTKNQVSLSC |
| | AVKGFYPSDIAVEWESNGQPENNYKTTPPVLDSDGSF |
| | FLVSKLTVDKSRWQQGNVFSCSVLHEALHNRFTQKSL |
| | SLSPG (SEQ ID NO: 61) |
| hu2G8.B6_G1AAA knob_QL | EVQLVQSGAEVKKPGASVKVSCKASGYTFTSYSIHWV |
| | RQAPGQGLEWIGRIDPISGGTLYNEMFKNWATLTVDV |
| | SISTAYMELSRLRSDDTAVYYCARFTMLVDHWGQGT |
| | TVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCDKTH |
| | TCPPCPAPEAAGAPSVFLFPPKPKDQLMISRTPEVTCV |
| | VVDVSHEDPEVKFNWYVDGVEVHNAKTKPREEQYNS |
| | TYRVVSVLTVLHQDWLNGKEYKCKVSNKALPAPIEK |
| | TISKAKGQPREPQVYTLPPCRDELTKNQVSLWCLVKG |
| | FYPSDIAVEWESNGQPENNYKTTPPVLDSDGSFFLYSK |
| | LTVDKSRWQQGNVFSCSVLHEALHNHYTQKSLSLSPG |
| | (SEQ ID NO: 62) |
| hu2G8.B6_LC | DVVMTQSPLSLPVTLGQPASISCKSSQSLLYSDGRTYL |
| | NWMQQRPGQSPRRLIYLVSKLDSGVPDRFSGSGSGTD |
| | FTLKISRVEAEDVGVYYCWQGTHFPQTFGGGTKVEIK |
| | RTVAAPSVFIFPPSDEQLKSGTASVVCLLNNFYPREAK |
| | VQWKVDNALQSGNSQESVTEQDSKDSTYSLSSTLTLS |
| | KADYEKHKVYACEVTHQGLSSPVTKSFNRGEC |
| | (SEQ ID NO: 63) |
| hu7D4.B4_1B10_G1AAA_hole_RF | EVQLVQSGAEVKKPGASVKVSCKVSGFSLTSYGILWV |
| | RQAPGQGLEWLGVVWPSGATNYNSALNSRLTITKDTS |
| | TSTVYMELRSLRSDDTAVYYCARWDYWGQGTTVTVS |
| | SASTKGPSVFPLAPSSKSTSGGTAALGCLVKDYFPEPV |
| | TVSWNSGALTSGVHTFPAVLQSSGLYSLSSVVTVPSSS |
| | LGTQTYICNVNHKPSNTKVDKKVEPKSCGGGGSEVKL |
| | LESGGGLVQPGGSLKLSCAASGFTFNTYAMNWVRQA |
| | PGKGLEWVGRIRSKANNYATYYADSVKGRFTISRDDS |
| | KNTLYLQMNNLKAEDTAVYYCVRHGNFGNSYVSWF |
| | AYWGQGTLVTVSSGGGGSGGGGSGGGGSAEAVVTQE |
| | PSLTVSPGGTVTLTCGSSTGAVTTSNYANWVQQKPGQ |
| | APRGLIGGTNKRAPGTPARFSGSLLGDKAALTITGAQP |
| | EDEAEYYCALWYSNLWVFGGGTKLTVLGGGGSDKT |
| | HTCPPCPAPEAAGAPSVFLFPPKPKDTLMISRTPEVTCV |
| | VVDVSHEDPEVKFNWYVDGVEVHNAKTKPREEQYNS |
| | TYRVVSVLTVLHQDWLNGKEYKCKVSNKALPAPIEK |
| | TISKAKGQPREPQVCTLPPSRDELTKNQVSLSCAVKGF |
| | YPSDIAVEWESNGQPENNYKTTPPVLDSDGSFFLVSKL |
| | TVDKSRWQQGNVFSCSVMHEALHNRFTQKSLSLSPG |
| | (SEQ ID NO: 64) |
| hu7D4.B4_1B4_G1AAA_hole_RF | EVQLVQSGAEVKKPGASVKVSCKVSGFSLTSYGILWV |
| | RQAPGQGLEWLGVVWPSGATNYNSALNSRLTITKDTS |
| | TSTVYMELRSLRSDDTAVYYCARWDYWGQGTTVTVS |
| | SASTKGPSVFPLAPSSKSTSGGTAALGCLVKDYFPEPV |
| | TVSWNSGALTSGVHTFPAVLQSSGLYSLSSVVTVPSSS |
| | LGTQTYICNVNHKPSNTKVDKKVEPKSCGGGGSEVKL |
| | VESGGGLVQPGGSLKLSCAASGFTFNTYAMNWVRQA |
| | PGKGLEWVGRIRSKYNNYATYYADSVKDRFTISRDDS |
| | QNILYLQMNNLRTEDTAVYYCVRHGNFGNSYVSWFA |
| | YWGQGTLVTVSSGGGGSGGGGSGGGGSAQAFVTQEP |
| | SLTVSPGGTVTLTCGSSTGAVTTSNYANWVQQKPGQA |
| | PRGLIGGTNKRAPGVPARFSGSLIGDKAALTITGAQPE |
| | DEAEYYCALWYSNLWVFGGGTKLTVLGGGGSDKTH |
| | TCPPCPAPEAAGAPSVFLFPPKPKDTLMISRTPEVTCVV |
| | VDVSHEDPEVKFNWYVDGVEVHNAKTKPREEQYNST |
| | YRVVSVLTVLHQDWLNGKEYKCKVSNKALPAPIEKTI |
| | SKAKGQPREPQVCTLPPSRDELTKNQVSLSCAVKGFY |
| | PSDIAVEWESNGQPENNYKTTPPVLDSDGSFFLVSKLT |
| | VDKSRWQQGNVFSCSVMHEALHNRFTQKSLSLSPG |
| | (SEQ ID NO: 65) |
| hu7D4.B4 G1AAA_knob | EVQLVQSGAEVKKPGASVKVSCKVSGFSLTSYGILWV |
| | RQAPGQGLEWLGVVWPSGATNYNSALNSRLTITKDTS |
| | TSTVYMELRSLRSDDTAVYYCARWDYWGQGTTVTVS |
| | SASTKGPSVFPLAPSSKSTSGGTAALGCLVKDYFPEPV |
| | TVSWNSGALTSGVHTFPAVLQSSGLYSLSSVVTVPSSS |
| | LGTQTYICNVNHKPSNTKVDKKVEPKSCDKTHTCPPC |
| | PAPEAAGAPSVFLFPPKPKDTLMISRTPEVTCVVVDVS |
| | HEDPEVKFNWYVDGVEVHNAKTKPREEQYNSTYRVV |
| | SVLTVLHQDWLNGKEYKCKVSNKALPAPIEKTISKAK |
| | GQPREPQVYTLPPCRDELTKNQVSLWCLVKGFYPSDI |
| | AVEWESNGQPENNYKTTPPVLDSDGSFFLYSKLTVDK |
| | SRWQQGNVFSCSVMHEALHNHYTQKSLSLSPG |
| | (SEQ ID NO: 66) |
| hu7D4.B4_1B10_G1AAA_hole_RF_QL | EVQLVQSGAEVKKPGASVKVSCKVSGFSLTSYGILWV |
| | RQAPGQGLEWLGVVWPSGATNYNSALNSRLTITKDTS |
| | TSTVYMELRSLRSDDTAVYYCARWDYWGQGTTVTVS |
| | SASTKGPSVFPLAPSSKSTSGGTAALGCLVKDYFPEPV |
| | TVSWNSGALTSGVHTFPAVLQSSGLYSLSSVVTVPSSS |
| | LGTQTYICNVNHKPSNTKVDKKVEPKSCGGGGSEVKL |
| | LESGGGLVQPGGSLKLSCAASGFTFNTYAMNWVRQA |
| | PGKGLEWVGRIRSKANNYATYYADSVKGRFTISRDDS |
| | KNTLYLQMNNLKAEDTAVYYCVRHGNFGNSYVSWF |
| | AYWGQGTLVTVSSGGGGSGGGGSGGGGSAEAVVTQE |
| | PSLTVSPGGTVTLTCGSSTGAVTTSNYANWVQQKPGQ |
| | APRGLIGGTNKRAPGTPARFSGSLLGDKAALTITGAQP |
| | EDEAEYYCALWYSNLWVFGGGTKLTVLGGGGSDKT |
| | HTCPPCPAPEAAGAPSVFLFPPKPKDQLMISRTPEVTC |
| | VVVDVSHEDPEVKFNWYVDGVEVHNAKTKPREEQY |
| | NSTYRVVSVLTVLHQDWLNGKEYKCKVSNKALPAPIE |
| | KTISKAKGQPREPQVCTLPPSRDELTKNQVSLSCAVKG |
| | FYPSDIAVEWESNGQPENNYKTTPPVLDSDGSFFLVSK |
| | LTVDKSRWQQGNVFSCSVLHEALHNRFTQKSLSLSPG |
| | (SEQ ID NO: 67) |
| hu7D4.B4_G1AAA_knob_QL | EVQLVQSGAEVKKPGASVKVSCKVSGFSLTSYGILWV |
| | RQAPGQGLEWLGVVWPSGATNYNSALNSRLTITKDTS |
| | TSTVYMELRSLRSDDTAVYYCARWDYWGQGTTVTVS |
| | SASTKGPSVFPLAPSSKSTSGGTAALGCLVKDYFPEPV |
| | TVSWNSGALTSGVHTFPAVLQSSGLYSLSSVVTVPSSS |
| | LGTQTYICNVNHKPSNTKVDKKVEPKSCDKTHTCPPC |
| | PAPEAAGAPSVFLFPPKPKDQLMISRTPEVTCVVVDVS |
| | HEDPEVKFNWYVDGVEVHNAKTKPREEQYNSTYRVV |
| | SVLTVLHQDWLNGKEYKCKVSNKALPAPIEKTISKAK |
| | GQPREPQVYTLPPCRDELTKNQVSLWCLVKGFYPSDI |
| | AVEWESNGQPENNYKTTPPVLDSDGSFFLYSKLTVDK |
| | SRWQQGNVFSCSVLHEALHNHYTQKSLSLSPG |
| | (SEQ ID NO: 68) |
| hu7D4.B4_LC | DIVMTQSPLSLPVTPGEPASISCRSSKSLLHSNGNTYLY |
| | WFLQKPGQSPQLLIYRVSNLASGVPDRFSGSGSGTDFT |
| | LKISRVEAEDVGVYYCMQHLEYPYTFGQGTKLEIKRT |
| | VAAPSVFIFPPSDEQLKSGTASVVCLLNNFYPREAKVQ |
| | WKVDNALQSGNSQESVTEQDSKDSTYSLSSTLTLSKA |
| | DYEKHKVYACEVTHQGLSSPVTKSFNRGEC |
| | (SEQ ID NO: 69) |

In one preferred embodiment, a multispecific antibody binds to LY6G6D and CD38, and comprises a first light chain subunit comprising SEQ ID NO: 57; a first heavy chain subunit comprising SEQ ID NO: 54; a second light chain subunit comprising SEQ ID NO: 57; and a second heavy chain subunit comprising SEQ ID NO: 56.

In one preferred embodiment, a multispecific antibody binds to LY6G6D and CD38, and comprises a first light chain subunit comprising SEQ ID NO: 57; a first heavy chain subunit comprising SEQ ID NO: 55; a second light chain subunit comprising SEQ ID NO: 57; and a second heavy chain subunit comprising SEQ ID NO: 56.

In one preferred embodiment, a multispecific antibody binds to LY6G6D and CD38, and comprises a first light chain subunit comprising SEQ ID NO: 57; a first heavy chain subunit comprising SEQ ID NO: 58; a second light chain subunit comprising SEQ ID NO: 57; and a second heavy chain subunit comprising SEQ ID NO: 60.

In one preferred embodiment, a multispecific antibody binds to LY6G6D and CD38, and comprises a first light chain subunit comprising SEQ ID NO: 57; a first heavy chain subunit comprising SEQ ID NO: 59; a second light chain subunit comprising SEQ ID NO: 57; and a second heavy chain subunit comprising SEQ ID NO: 60.

In one preferred embodiment, a multispecific antibody binds to LY6G6D and CD38, and comprises a first light chain subunit comprising SEQ ID NO: 57; a first heavy chain subunit comprising SEQ ID NO: 61; a second light chain subunit comprising SEQ ID NO: 57; and a second heavy chain subunit comprising SEQ ID NO: 62.

In one preferred embodiment, a multispecific antibody binds to LY6G6D and CD38, and comprises a first light chain subunit comprising SEQ ID NO: 69; a first heavy chain subunit comprising SEQ ID NO: 64; a second light chain subunit comprising SEQ ID NO: 69; and a second heavy chain subunit comprising SEQ ID NO: 66.

In one preferred embodiment, a multispecific antibody binds to LY6G6D and CD38, and comprises a first light chain subunit comprising SEQ ID NO: 69; a first heavy chain subunit comprising SEQ ID NO: 65; a second light chain subunit comprising SEQ ID NO: 69; and a second heavy chain subunit comprising SEQ ID NO: 66.

In one preferred embodiment, a multispecific antibody binds to LY6G6D and CD38, and comprises a first light chain subunit comprising SEQ ID NO: 69; a first heavy chain subunit comprising SEQ ID NO: 67; a second light chain subunit comprising SEQ ID NO: 69; and a second heavy chain subunit comprising SEQ ID NO: 68.

Various formats of multi-specific antibodies are within the ambit of the invention, including, without limitation, single chain polypeptides, two chain polypeptides, three chain polypeptides, four chain polypeptides, and multiples thereof. The multi-specific antibodies herein specifically include T-cell multi-specific (e.g., bispecific) antibodies binding to LY6G6D and CD38 (anti-LY6G6D×anti-CD3& antibodies). Such antibodies induce potent T-cell mediated killing of cells expressing LY6G6D.

### Preparation of Anti-LY6G6D Antibodies

The antibodies of the present invention can be prepared by methods known in the art, such as recombinant DNA technology, by expression of the encoding nucleic acid in a suitable eukaryotic or prokaryotic host, including, for example, mammalian cells (e.g., CHO cells), E. coli or yeast.

Antibodies binding to non-overlapping epitopes on a LY6G6D protein can be identified by competition binding assays, such as enzyme-linked immunoassays (ELISA assays) or flow cytometric competitive binding assays. For example, one can use competition between known antibodies binding to the target antigen and the antibody of interest. By using this approach, one can divide a set of antibodies into those that compete with the reference antibody and those that do not. The non-competing antibodies are identified as binding to a distinct epitope that does not overlap with the epitope bound by the reference antibody. Often, one antibody is immobilized, the antigen is bound, and a second, labeled (e.g., biotinylated) antibody is tested in an ELISA assay for ability to bind the captured antigen. This can be performed also by using surface plasmon resonance (SPR) platforms, including ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc).

Typically, an antibody “competes” with a reference antibody if it causes about 15-100% reduction in the binding of the reference antibody to the target antigen, as determined by standard techniques, such as by the competition binding assays described above. In various embodiments, the relative inhibition is at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95% or higher.

### Pharmaceutical Compositions, Uses and Methods of Treatment

It is another aspect of the present invention to provide pharmaceutical compositions comprising one or more antibodies of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

In one embodiment, a pharmaceutical composition comprises an antibody that binds to LY6G6D. In another embodiment, a pharmaceutical composition comprises a multi-specific (including bispecific) antibody with binding specificity for two or more non-overlapping epitopes on an LY6G6D protein. In a preferred embodiment, a pharmaceutical composition comprises a multi-specific (including bispecific) antibody that binds to LY6G6D and a binding target on an effector cell (e.g., a binding target on a T-cell, such as, e.g., a CD38 protein on a T-cell).

Pharmaceutical compositions of the antibodies used in accordance with the present invention are prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions.

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The antibodies herein can be administered by intravenous injection or infusion or subcutaneously. For injection administration, the antibodies herein can be formulated in aqueous solutions, preferably in physiologically-compatible buffers.

Antibody formulations are disclosed, for example, in U.S. Pat. No. 9,034,324. Similar formulations can be used for the heavy chain antibodies, including UniAbs™, of the present invention. Subcutaneous antibody formulations are described, for example, in US20160355591 and US20160166689.

### Methods of Use

The anti-LY6G6D antibodies and pharmaceutical compositions described herein can be used for the treatment of diseases and conditions characterized by the expression of LY6G6D, including, without limitation, the conditions and diseases described further herein.

LY6G6D belongs to a cluster of leukocyte antigen-6 (LY6) genes located in the major histocompatibility complex (MHC) class III region on chromosome 6. Members of the LY6 superfamily typically contain 70 to 80 amino acids, including 8 to 10 cysteines. Most LY6 proteins are attached to the cell surface by a glycosylphosphatidylinositol (GPI) anchor that is directly involved in signal transduction.

RNA expression analysis from TCGA databases shows that LY6G6D is highly expressed and specific to colorectal cancer. Expression on other tumor types and normal cells is negligible, making it a highly favorable tumor target. Therapeutic development of antibodies against this target may therefore be efficacious in treating certain cohorts of colorectal cancer patients that are refractory to chemotherapy or immune checkpoint therapy.

In one aspect, the anti-LY6G6D antibodies and pharmaceutical compositions herein can be used to treat disorders characterized by the expression of LY6G6D, including, without limitation, colorectal cancer. In one preferred embodiment, the colorectal cancer is refractory colorectal cancer, wherein the patient receiving treatment has previously received another therapy (e.g., a chemotherapy regimen) and has not achieved remission of the colorectal cancer. In one preferred embodiment, the colorectal cancer is refractory colorectal cancer, wherein the patient receiving treatment has previously received another therapy (e.g., a checkpoint inhibitor therapy) and has not achieved remission of the colorectal cancer.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Typically, compositions are prepared as injectables, either as liquid solutions or suspensions; solid forms suitable for solution in, or suspension in, liquid vehicles prior to injection can also be prepared. The pharmaceutical compositions herein are suitable for intravenous or subcutaneous administration, directly or after reconstitution of solid (e.g., lyophilized) compositions. The preparation also can be emulsified or encapsulated in liposomes or micro particles such as poly lactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the antibodies and antibody structures described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the antibodies described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents and formulations thereof, of the invention and instructions for use. The kit can further contain a least one additional reagent. e.g., a chemotherapeutic drug, a checkpoint inhibitor, etc. Kits typically include a label indicating the intended use of the contents of the kit. The term “label” as used herein includes any writing, or recorded material supplied on or with a kit, or which otherwise accompanies a kit.

Aspects of the invention include diagnostic assay methods, e.g., diagnostic immunoassays, which can be used to detect the presence or absence of LY6G6D in a test sample. The immunoassay format used for the detection of LY6G6D can be configured in a variety of ways. The immunoassays can include both homogeneous and heterogeneous assays, competitive and non-competitive assays, direct and indirect assays, and “sandwich” assays. Useful assay formats include, but are not limited to, enzyme immunoassays, for example, enzyme linked immunosorbent assays (ELISA), chemiluminescent immune-assays (CLIA), electrochemiluminescent assays, radioimmunoassays, immunofluorescence assays, fluorescence anisotropy assays, immunoprecipitation assays, equilibrium dialysis assays, immunodiffusion assays, immunoblotting assays, agglutination assays, luminescent proximity assays, and nephelometry assays.

Regardless of the assay format used, a biological sample is contacted with an anti-LY6G6D antibody of the present invention. In some embodiments, the biological sample can be immobilized on a solid support, e.g., a histology slide. In some embodiments, the biological sample is contacted with an anti-LY6G6D antibody of the invention that has been immobilized on a solid support. The solid support can be, for example, a plastic surface, a glass surface, a paper or fibrous surface, or the surface of a particle. More specifically, the support can include a microplate, a bead, a polyvinylidene difluoride (PVDF) membrane, a nitrocellulose membrane, a nylon membrane, a porous membrane, a non-porous membrane, or any equivalent thereof. The composition of the substrate can be varied. For example, substrates or support can comprise glass, cellulose-based materials, thermoplastic polymers, such as polyethylene, polypropylene, or polyester, sintered structures composed of particulate materials (e.g., glass or various thermoplastic polymers), or cast membrane film composed of nitrocellulose, nylon, or polysulfone. In general embodiments, the substrate may be any surface or support upon which an antibody or a polypeptide can be immobilized, including one or more of a solid support (e.g., glass, such as a glass slide or a coated plate, silica, plastic or derivatized plastic, paramagnetic or non-magnetic metal), a semi-solid support (e.g., a polymeric material, a gel, agarose, or other matrix), and/or a porous support (e.g., a filter, a nylon or nitrocellulose membrane or other membrane). In some embodiments, synthetic polymers can be used as a substrate, including, e.g., polystyrene, polypropylene, polyglycidylmethacrylate, aminated or carboxylated polystyrenes, polyacrylamides, polyamides, and polyvinylchlorides.

In some embodiments, an immunoassay format can be a two antibody “sandwich” assay. The biological sample is contacted with an anti-LY6G6D antibody of the invention that has been immobilized on a solid support, for example, a microtiter plate. The sample and the first antibody are incubated under conditions that favor specific binding and the formation of an LY6G6D-antibody complex. Following the contacting step, unbound constituents of the biological sample are removed. Then, the complex is contacted with a second antibody. In some embodiments, the second antibody is an anti-LY6G6D antibody. The second antibody can bind to a different LY6G6D epitope than the epitope bound by the first antibody. Thus, the first and second antibodies do not competitively inhibit one another for binding to LY6G6D. In some embodiments, the second antibody can bind to a portion of the first antibody to facilitate detection.

Antibody binding can be measured in a variety of ways. The signal, for example, generated by a detectable label, can be analyzed, and if applicable, quantified using an optical scanner or other image acquisition device and software that permits the measurement of the signal, for example, a fluorescent signal, a luminescent signal, or a phosphorescent signal, or a radioactive signal, associated with complex formation. Exemplary instrumentation for measuring a detectable signal can include, but is not limited to microplate readers, fluorimeters, spectrophotometers, and gamma counters.

The level of LY6G6D in a biological sample can be compared with that of a reference sample. Standard reference levels typically represent the average LY6G6D levels derived from a population of individuals. The reference population may include individuals of similar age, body size, ethnic background or general health as the individual in question. Thus, the LY6G6D levels in a patient's sample can be compared to values derived from: 1) individuals who are known to have a colorectal cancer and who express LY6G6D and whose bodily fluids contain LY6G6D; 2) individuals who do not have a colorectal cancer and whose bodily fluids contain low levels of LY6G6D.

In some embodiments, a reference chart can be used to determine whether or not a particular level of LY6G6D in a sample is elevated relative to a control sample or a larger population. For example, a reference chart can contain the normal range of LY6G6D found in healthy individuals of the same age, ethnic background or general health as the individual in question. Using this reference chart, any level of LY6G6D measured in a sample can be classified as being low, normal, or elevated relative to a control sample or relative to an average value derived from a larger population. The term “elevated level” is defined as a level, which is higher, preferably at least 2% higher, more preferably at least 5% higher, than a reference level.

Alternatively, or in addition, the level of LY6G6D in a biological sample can be “normalized” against the level of one or more additional biological markers, for example, another marker whose expression is independent of LY6G6D expression. That is, the levels of the additional marker can be evaluated in parallel with those of LY6G6D, either at the same time or on a separate occasion. The additional marker can serve as an internal control for sample preparation, handling and storage as well as day-to-day assay variability. The values for the level LY6G6D and the additional marker may be expressed as a ratio and the ratio may be compared to a similar ratio obtained for a reference sample or population. A useful second marker can be alpha-fetoprotein.

The diagnostic methods disclosed herein are useful in the detection of a colorectal cancer in a patient suspected of having or at risk for colorectal cancer. The methods can also be used in the analysis of samples from a patient who has been treated for colorectal cancer in order to determine whether the patient is at risk for experiencing relapsed or refractory colorectal cancer. The methods can also be used for monitoring the course of the treatment, for example treatment with a therapeutic agent such as a chemotherapy, checkpoint inhibitor therapy, radiation therapy or surgery, to determine the efficacy of the treatment and to allow the managing clinician to alter the treatment, if needed. The methods may also be used in the detection, monitoring, or analysis of a patient suffering from or at risk for any disorder that is associated with a modulation, for example an increase, in the level of LY6G6D in a biological sample, for example, a blood or serum sample, obtained from the patient.

The compositions described herein can be packaged in suitable containers labeled, for example, for use in the detection, identification, and quantification of LY6G6D in a biological sample. The articles of manufacture, also referred to as “kits” or “assay kits”, may include antibodies, antigen binding fragments of antibodies, or any combination thereof, of the present invention, as well as media, purified samples of antigen for use as positive controls, or any combination thereof. The containers included in the kits can include a composition comprising an antibody of the present invention that specifically or preferentially binds to LY6G6D. Suitable buffers for diluting or reconstituting test samples and antibodies may also be provided. Some of the components may be provided in dry form, and may require reconstitution. The anti-LY6G6D antibodies can be pre-bound to an assay device, for example, a microplate. Thus, in one embodiment, a kit for the detection, identification and quantification of LY6G6D comprises an anti-LY6G6D antibody pre-bound to an assay device. The kit may optionally further comprise a detectable label.

Accordingly, packaged products (e.g., sterile containers containing one or more of the compositions described herein and packaged for storage, shipment, or sale at concentrated or ready-to-use concentrations) and kits, including at least one composition of the invention, e.g., an anti-LY6G6D antibody, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which diagnosis or treatment is required.

Reagents for particular types of assays can also be provided in kits of the invention. Thus, the kits can include a population of beads (e.g., suitable for an agglutination assay or a lateral flow assay), or a plate (e.g., a plate suitable for an ELISA assay). In other embodiments, the kits comprise a device, such as a lateral flow immunoassay device, an analytical rotor, or an electrochemical, optical, or opto-electronic sensor. The population of beads, the plate, and the devices are useful for performing an immunoassay. For example, they can be useful for detecting formation of a first agent-analyte-second agent complex.

In addition, the kits can include various diluents and buffers, labeled conjugates or other agents for the detection of specifically bound antigens or antibodies, and other signal-generating reagents, such as enzyme substrates, cofactors and chromogens. The kits can include one or more reference samples of varying concentrations, for example, purified recombinant LY6G6D. The kits can also include a positive control, for example a cell supernatant from a cell line that over expresses LY6G6D. Other components of a kit can include coating reagents, polyclonal or monoclonal capture antibodies specific for an antigen or analyte to be tested, or a cocktail of two or more of the antibodies, purified or semi-purified extracts of these antigens as standards, monoclonal antibody detector antibodies, an anti-mouse, anti-dog, anti-chicken, or anti-human antibody with indicator molecule conjugated thereto, indicator charts for colorimetric comparisons, disposable gloves, decontamination instructions, applicator sticks or containers, a sample preparatory cup, etc. In one embodiment, a kit comprises buffers or other reagents appropriate for constituting a reaction medium allowing the formation of a peptide-antibody complex.

Such kits provide a convenient, efficient way for a clinician to determine whether subject has or is at risk for colorectal cancer. Thus, in certain embodiments, the kits further comprise instructions for use. The product may also include a legend (e.g., a printed label or insert or other medium describing the product's use (e.g., an audio- or videotape)). The legend can be associated with the container (e.g., affixed to the container) and can describe the manner in which the assay should be performed, indications therefor, and other uses.

The invention now being fully described, it will be apparent to one of ordinary skill in the art that various changes and modifications can be made without departing from the spirit or scope of the invention.

### EXAMPLES

### Example 1: Binding Analysis of Humanized Anti-LY6G6D Antibodies Against LY6G6D-Expressing Cell Lines and Primary Cells

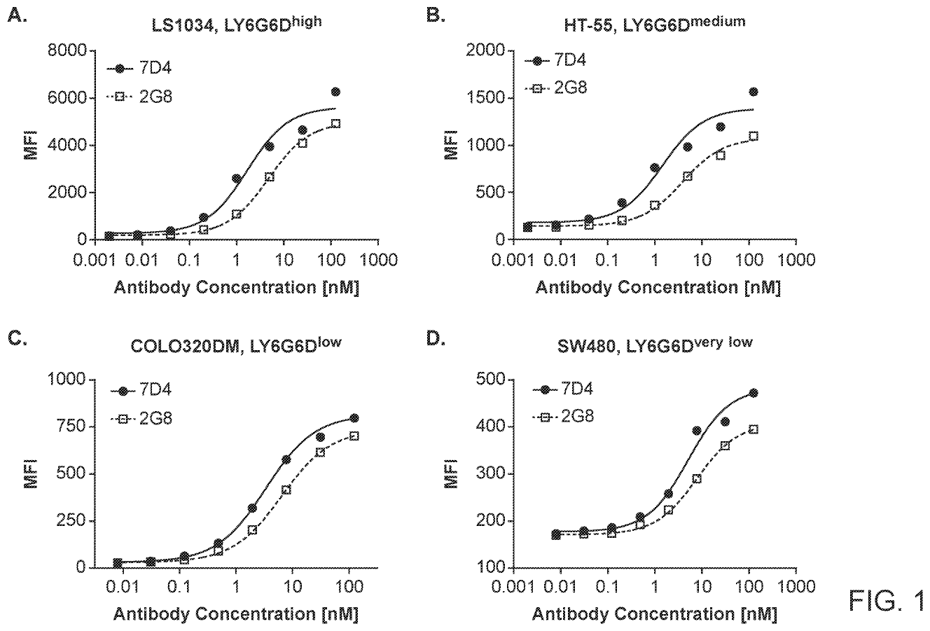

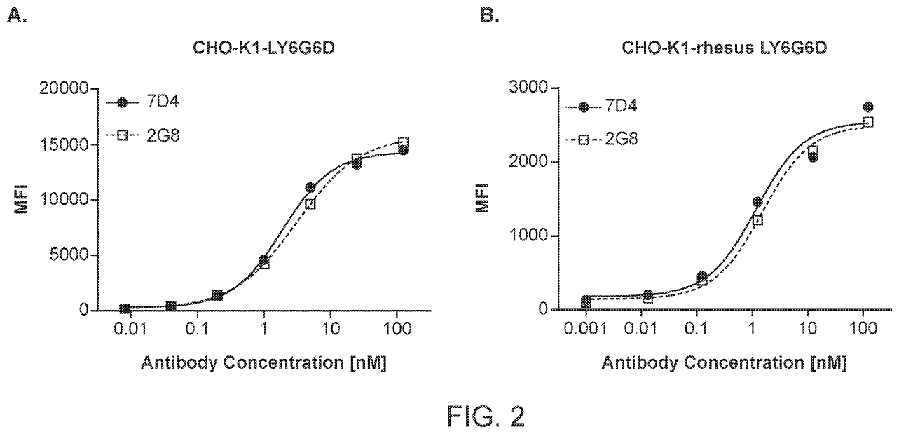

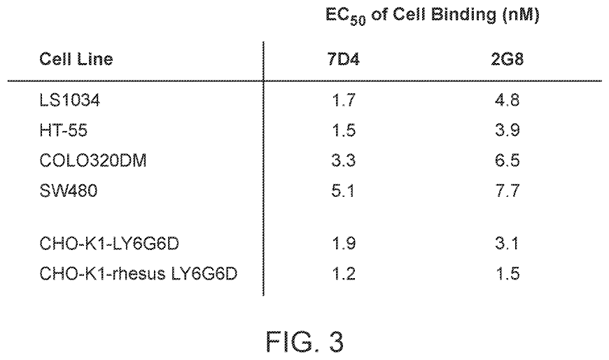

Humanized anti-LY6G6D antibodies were tested for their binding to cells expressing LY6G6D. Four colon cancer cell lines expressing different levels of LY6G6D were tested, along with CHO-K1 cells expressing human and rhesus LY6G6D. Cells were washed with and resuspended in FACS buffer. Cells were plated onto 96 well plates at 1E5 cells/well. Cells were stained with titrated anti-LY6G6D antibodies on ice for 30 minutes followed by washing and secondary staining with 1:500 diluted goat anti-human IgG-AF647. Cells were analyzed by flow cytometry following final washes and addition of 7-AAD. The geometric means of fluorescence intensity were graphed against the concentration of antibodies to generate a dose response curve. FIG. 1 shows the binding curves for colon cancer cell lines. FIG. 2 shows the binding curves for CHO-K1 cells. FIG. 3 summarizes the EC₅₀ of cell binding.

### Example 2: Binding of LY6G6D×CD3 Bispecific Antibodies to Colon Cancer Cell Lines and T-Cells

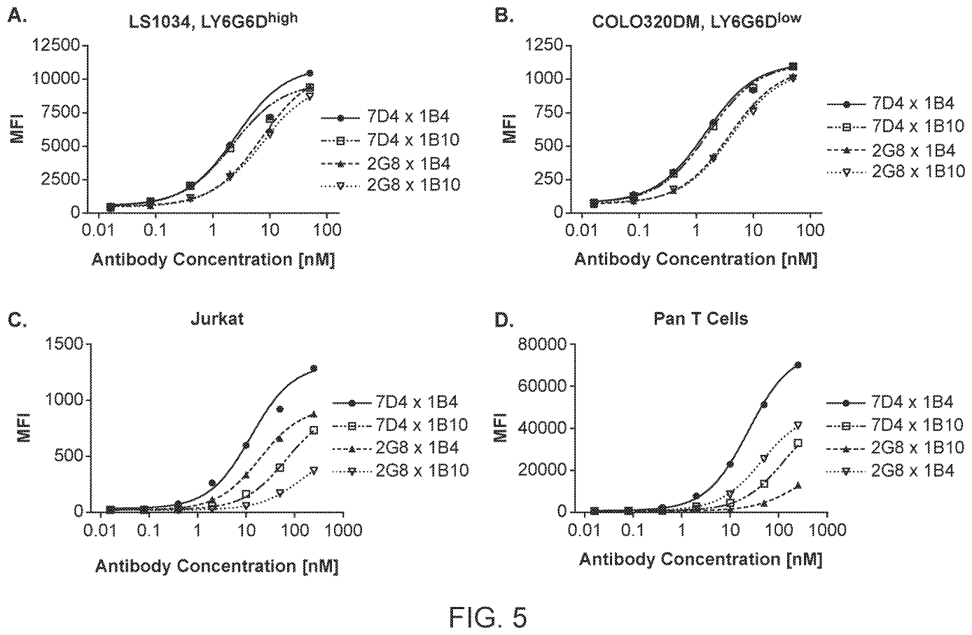

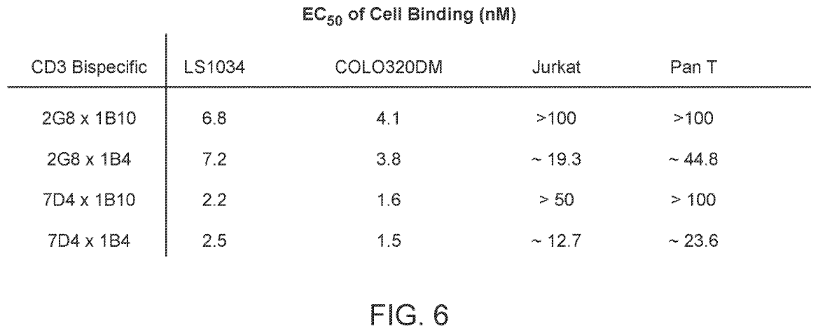

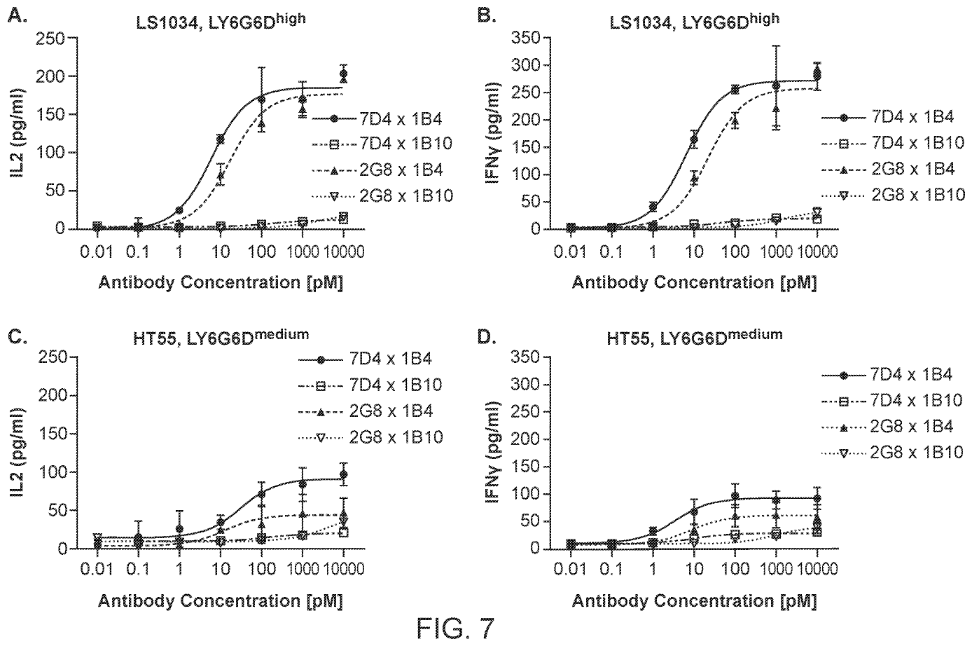

LY6G6D×CD3 antibodies were tested for their binding to colon cancer cells expressing LY6G6D and T-cells expressing CD3. Cells were washed with and resuspended in FACS buffer. Cells were plated onto 96 well plates at 1E5 cells/well. Cells were stained with titrated anti-LY6G6D antibodies on ice for 30 minutes, followed by washing and secondary staining with 1:500 diluted goat anti-human IgG-AF647. Cells were analyzed by flow cytometry following final washes and addition of 7-AAD. The geometric means of fluorescence intensity were graphed against the concentration of antibodies to generate a dose response curve. FIG. 5 shows the binding curves. FIG. 6 summarizes the EC₅₀ of cell binding.

### Example 3: Cytokine Release Mediated by LY6G6D×CD3 Bispecific Antibodies in the Presence of Colon Cancer Cell Lines

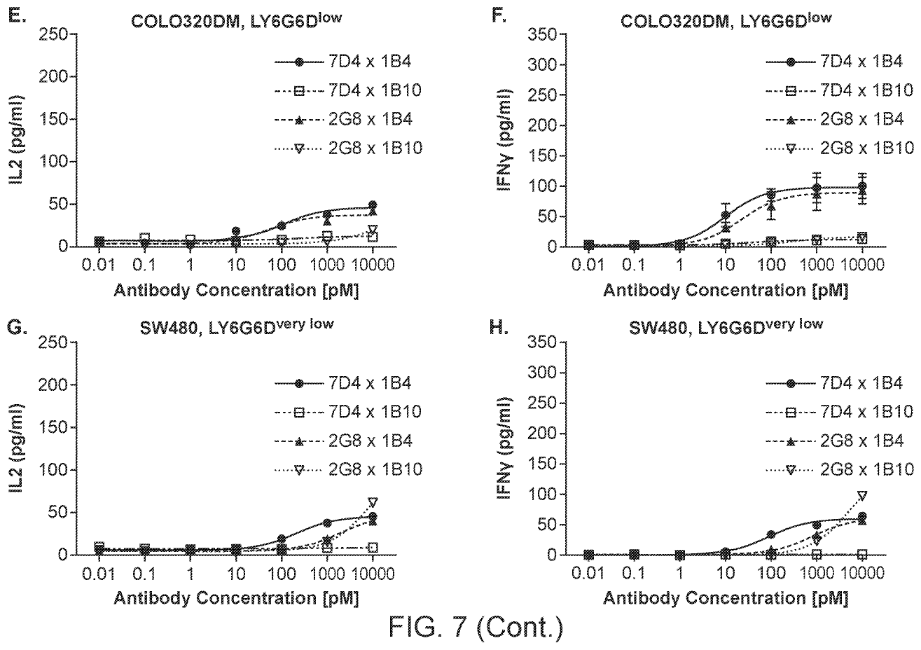

LY6G6D×CD3 bispecific antibodies were tested in a cytokine release assay. Pan T-cells were isolated from Human PBMCs and incubated with LY6G6D×CD3 bispecific antibodies in the presence of colon cancer cells expressing LY6G6D. Supernatant was collected after 48 h for IL-2 and IFNγ release detection using ELISA kits from R&D Systems. The data are shown in FIG. 7.

### Example 4: Activation of CD8+ T-Cells Mediated by LY6G6D×CD3 Bispecific Antibodies

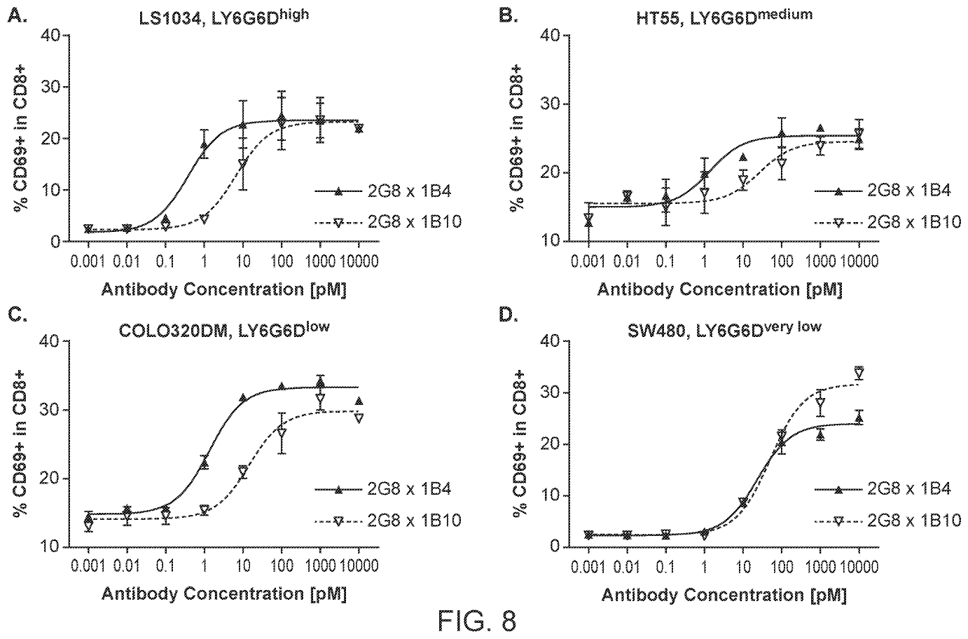

LY6G6D×CD3 bispecific antibodies were tested for their ability to activate T-cells in the presence of colon cancer cells. Pan T-cells were isolated from PBMCs and co-cultured with colon cancer cell lines and LY6G6D×CD3 bispecific antibodies for 48 hours. Cells were harvested and stained for CD4, CD8, CD25, CD69 and with 7-AAD cell viability dye. The percentage of CD4+ and CD8+ T-cells expressing CD25 and CD69 was plotted against the concentration of antibodies. The results are shown in FIG. 8.

### Example 5: Cytotoxicity of Colon Cancer Cells Mediated by LY6G6D×CD3 Bispecific Antibodies

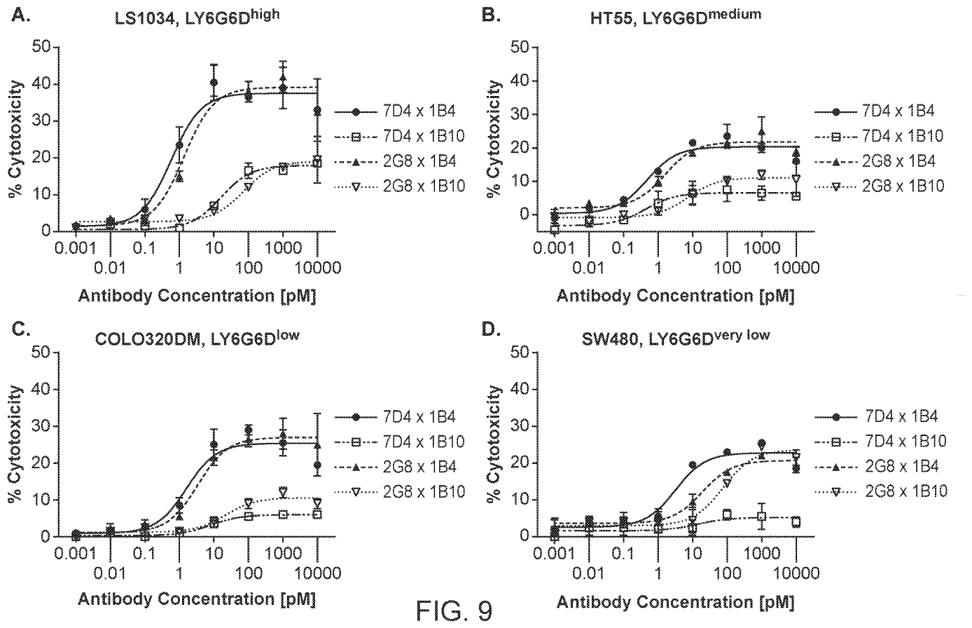

LY6G6D×CD3 bispecific antibodies were tested for their potency in mediating killing of colon cancer cells. Pan T-cells were isolated from PBMCs and co-cultured with colon cancer cell lines and LY6G6D×CD3 bispecific antibodies for 48 hours. Cytotoxicity was determined using CytotoxGlo kit from Promega. The results are shown in FIG. 9.

### Example 6: In Vivo Tumor Growth Inhibition of HT55 Colon Cancer Xenograft

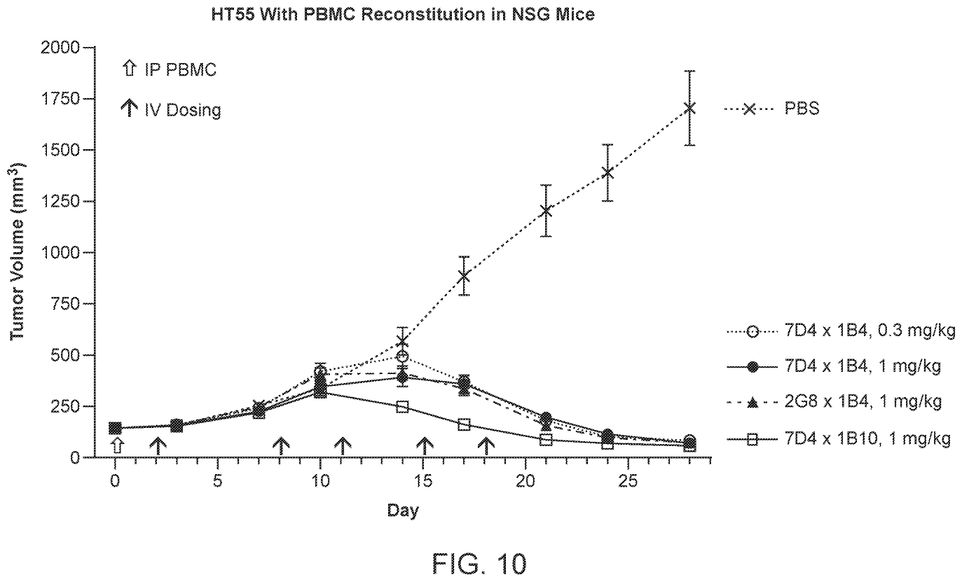

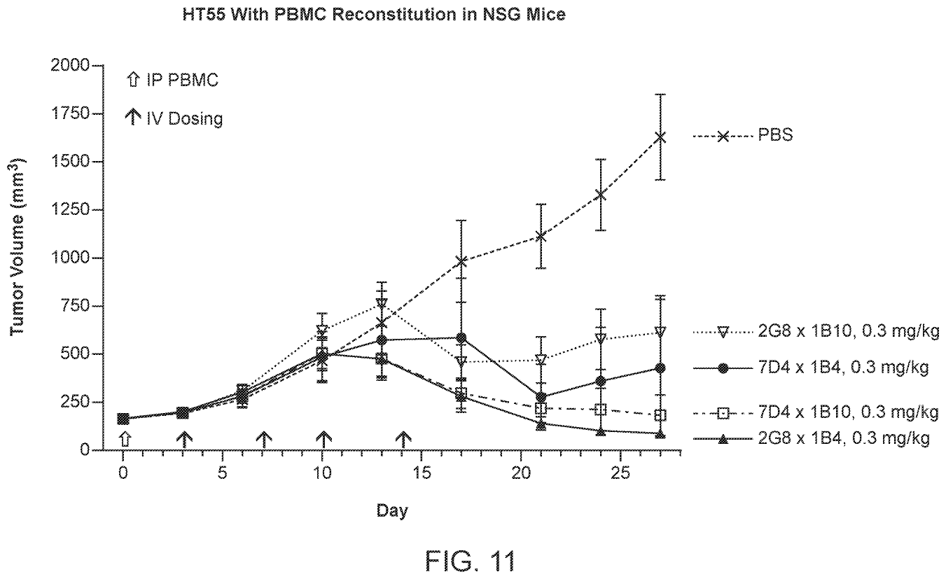

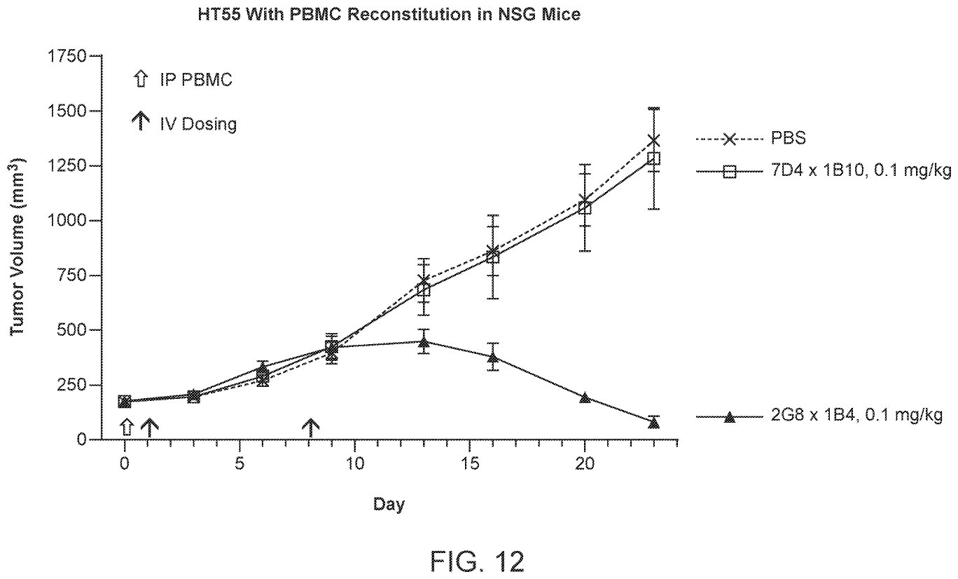

LY6G6D×CD3 bispecific antibodies were tested for their ability to suppress the growth of HT55 colon cancer cells in vivo. In a human PBMC reconstitution model, HT55 cells were implanted subcutaneously in the flanks of NSG mice and let grow until around 150 mm3, after which human PBMCs were injected intraperitoneally. After 24-72 hours, LY6G6D×CD3 bispecific antibodies were dosed intravenously. FIG. 10 shows tumor growth inhibition after 0.3-1 mg/kg biweekly dosing. FIG. 11 shows tumor growth inhibition after 0.3 mg/kg biweekly dosing. FIG. 12 shows tumor growth inhibition after 0.1 mg/kg weekly dosing.

### Example 7: In Vivo Tumor Growth Inhibition of COLO320DM Colon Cancer Xenograft

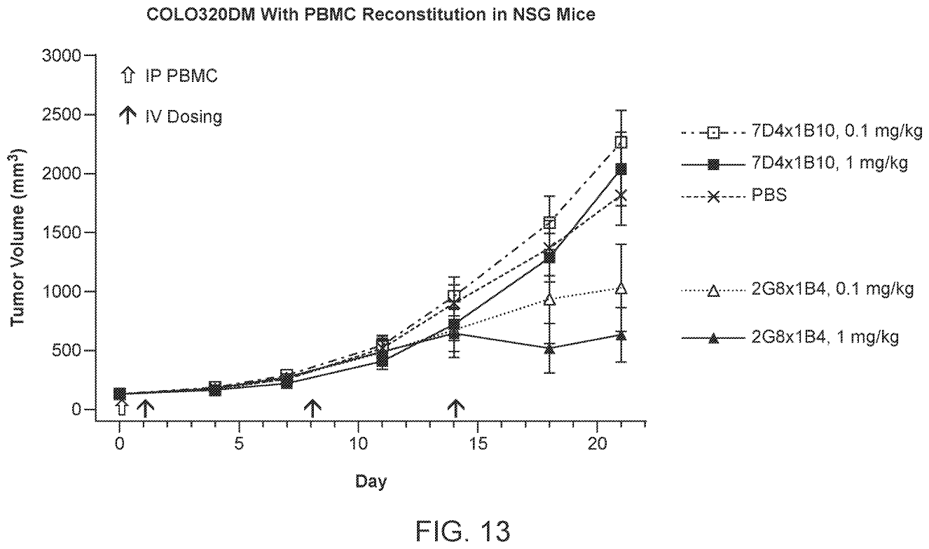

LY6G6D×CD3 bispecific antibodies were tested for their ability to suppress the growth of COL032DM colon cancer cells in vivo. In a human PBMC reconstitution model, COLO320DM cells were implanted subcutaneously in the flanks of NSG mice and let grow until around 150 mm3, after which human PBMC were injected intraperitoneally. After 24 hours, LY6G6D×CD3 bispecific antibodies were dosed weekly intravenously at 0.1-1 mg/kg. The results are shown in FIG. 13.

### Example 8: PK of LY6G6D×CD3 Bispecific Antibody with Wild Type FcRn Binding and T250Q M428L Mutations in Tg276 Hemizygous Human FcRn Transgenic Mice

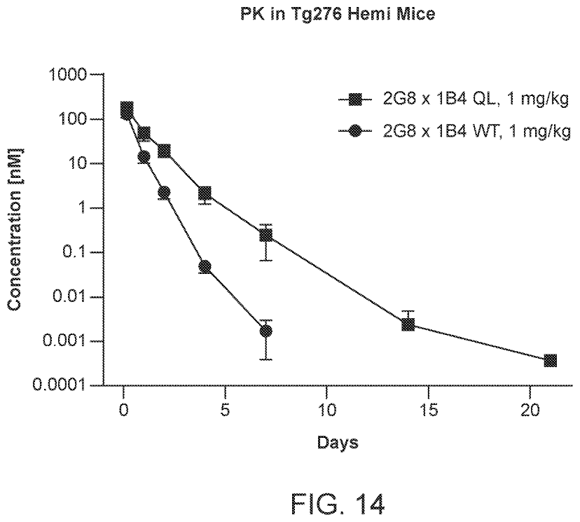

The pharmacokinetics of an LY6G6D×CD3 bispecific antibody with (QL) or without (WT) FcRn binding mutations were assessed in transgenic mice expressing human FcRn. A single dose of the antibodies was administered, and serum samples were collected at various time points until day 21. An ELISA was used to determine the serum concentration of the antibodies at each time point. The results are shown in FIG. 14.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An antibody that binds to LY6G6D, comprising a first binding unit comprising:
a heavy chain variable region comprising:
(a) a CDRH1 sequence of any one of SEQ ID NOs: 1-6; and/or
(b) a CDRH2 sequence of any one of SEQ ID NOs: 7-11; and/or
(c) a CDRH3 sequence of any one of SEQ ID NOs: 12-15; and
a light chain variable region comprising:
(d) a CDRL1 sequence of any one of SEQ ID NOs: 16-19; and/or
(e) a CDRL2 sequence of any one of SEQ ID NOs: 20-23; and/or
(f) a CDRL3 sequence of any one of SEQ ID NOs: 24-26.

2. The antibody of claim 1, wherein the CDRH1, CDRH2 and CDRH3 sequences of the first binding unit are present in a human VH framework.

3. The antibody of claim 1 or 2, wherein the CDRL1, CDRL2 and CDRL3 sequences of the first binding unit are present in a human VL framework.

4. The antibody of any one of claims 1-3, wherein the first binding unit comprises:
a heavy chain variable region comprising:
(a) a CDRH1 sequence of any one of SEQ ID NOs: 1-6; and
(b) a CDRH2 sequence of any one of SEQ ID NOs: 7-11; and
(c) a CDRH3 sequence of any one of SEQ ID NOs: 12-15.

5. The antibody of claim 4, wherein the first binding unit comprises:
a heavy chain variable region comprising:
(a) a CDRH1 sequence of SEQ ID NO: 1, a CDRH2 sequence of SEQ ID NO: 7, and a CDRH3 sequence of SEQ ID NO: 12; or
(b) a CDRH1 sequence of SEQ ID NO: 2, a CDRH2 sequence of SEQ ID NO: 8, and a CDRH3 sequence of SEQ ID NO: 13; or
(c) a CDRH1 sequence of SEQ ID NO: 3, a CDRH2 sequence of SEQ ID NO: 9, and a CDRH3 sequence of SEQ ID NO: 13; or
(d) a CDRH1 sequence of SEQ ID NO: 4, a CDRH2 sequence of SEQ ID NO: 10, and a CDRH3 sequence of SEQ ID NO: 14; or
(e) a CDRH1 sequence of SEQ ID NO: 5, a CDRH2 sequence of SEQ ID NO: 11, and a CDRH3 sequence of SEQ ID NO: 15; or
(f) a CDRH1 sequence of SEQ ID NO: 6, a CDRH2 sequence of SEQ ID NO: 11, and a CDRH3 sequence of SEQ ID NO: 15.

6. The antibody of any one of claims 1-5, wherein the first binding unit comprises:
a light chain variable region comprising:
(a) a CDRL1 sequence of any one of SEQ ID NOs: 16-19; and
(b) a CDRL2 sequence of any one of SEQ ID NOs: 20-23; and
(c) a CDRL3 sequence of any one of SEQ ID NOs: 24-26.

7. The antibody of claim 6, wherein the first binding unit comprises:
a light chain variable region comprising:
(a) a CDRL1 sequence of SEQ ID NO: 16, a CDRL2 sequence of SEQ ID NO: 20, and a CDRL3 sequence of SEQ ID NO: 24; or
(b) a CDRL1 sequence of SEQ ID NO: 17, a CDRL2 sequence of SEQ ID NO: 21, and a CDRL3 sequence of SEQ ID NO: 25: or
(c) a CDRL1 sequence of SEQ ID NO: 18, a CDRL2 sequence of SEQ ID NO: 22, and a CDRL3 sequence of SEQ ID NO: 24; or
(d) a CDRL1 sequence of SEQ ID NO: 19, a CDRL2 sequence of SEQ ID NO: 23, and a CDRL3 sequence of SEQ ID NO: 26.

8. The antibody of any one of claims 1-7, wherein the first binding unit comprises:
(a) a heavy chain variable region comprising:
a CDRH1 sequence of SEQ ID NO: 1, a CDRH2 sequence of SEQ ID NO: 7, and a CDRH3 sequence of SEQ ID NO: 12; and
a light chain variable region comprising:
a CDRL1 sequence of SEQ ID NO: 16, a CDRL2 sequence of SEQ ID NO: 20, and a CDRL3 sequence of SEQ ID NO: 24; or
(b) a heavy chain variable region comprising:
a CDRH1 sequence of SEQ ID NO: 2, a CDRH2 sequence of SEQ ID NO: 8, and a CDRH3 sequence of SEQ ID NO: 13; and
a light chain variable region comprising:
a CDRL1 sequence of SEQ ID NO: 17, a CDRL2 sequence of SEQ ID NO: 21, and a CDRL3 sequence of SEQ ID NO: 25; or
(c) a heavy chain variable region comprising:
a CDRH1 sequence of SEQ ID NO: 3, a CDRH2 sequence of SEQ ID NO: 9, and a CDRH3 sequence of SEQ ID NO: 13; and
a light chain variable region comprising:
a CDRL1 sequence of SEQ ID NO: 17, a CDRL2 sequence of SEQ ID NO: 21, and a CDRL3 sequence of SEQ ID NO: 25; or
(d) a heavy chain variable region comprising:
a CDRH1 sequence of SEQ ID NO: 4, a CDRH2 sequence of SEQ ID NO: 10, and a CDRH3 sequence of SEQ ID NO: 14; and
a light chain variable region comprising:
a CDRL1 sequence of SEQ ID NO: 18, a CDRL2 sequence of SEQ ID NO: 22, and a CDRL3 sequence of SEQ ID NO: 24; or
(e) a heavy chain variable region comprising:
a CDRH1 sequence of SEQ ID NO: 5, a CDRH2 sequence of SEQ ID NO: 11, and a CDRH3 sequence of SEQ ID NO: 15; and
a light chain variable region comprising:
a CDRL1 sequence of SEQ ID NO: 19, a CDRL2 sequence of SEQ ID NO: 23, and a CDRL3 sequence of SEQ ID NO: 26: or
(f) a heavy chain variable region comprising:
a CDRH1 sequence of SEQ ID NO: 6, a CDRH2 sequence of SEQ ID NO: 11, and a CDRH3 sequence of SEQ ID NO: 15; and
a light chain variable region comprising:
a CDRL1 sequence of SEQ ID NO: 19, a CDRL2 sequence of SEQ ID NO: 23, and a CDRL3 sequence of SEQ ID NO: 26.

9. The antibody of claim 8, wherein the first binding unit comprises a heavy chain variable region sequence having at least 95% identity to any one of SEQ ID NOs: 27-34.

10. The antibody of claim 8, wherein the first binding unit comprises a heavy chain variable region sequence comprising any one of SEQ ID NOs: 27-34.

11. The antibody of any one of claims 8-10, wherein the first binding unit comprises a light chain variable region sequence having at least 95% identity to any one of SEQ ID NOs: 35-42.

12. The antibody of any one of claims 8-10, wherein the first binding unit comprises a light chain variable region sequence comprising any one of SEQ ID NOs: 35-42.

13. The antibody of any one of claims 8-12, wherein the first binding unit comprises:
(a) a heavy chain variable region sequence of SEQ ID NO: 27 and a light chain variable region sequence of SEQ ID NO: 35; or
(b) a heavy chain variable region sequence of SEQ ID NO: 28 and a light chain variable region sequence of SEQ ID NO: 36; or
(c) a heavy chain variable region sequence of SEQ ID NO: 29 and a light chain variable region sequence of SEQ ID NO: 37; or
(d) a heavy chain variable region sequence of SEQ ID NO: 30 and a light chain variable region sequence of SEQ ID NO: 38; or
(e) a heavy chain variable region sequence of SEQ ID NO: 31 and a light chain variable region sequence of SEQ ID NO: 39; or
(f) a heavy chain variable region sequence of SEQ ID NO: 32 and a light chain variable region sequence of SEQ ID NO: 40; or
(g) a heavy chain variable region sequence of SEQ ID NO: 33 and a light chain variable region sequence of SEQ ID NO: 41; or
(h) a heavy chain variable region sequence of SEQ ID NO: 34 and a light chain variable region sequence of SEQ ID NO: 42.

14. The antibody of any one of claims 1-13, further comprising a heavy chain constant region.

15. The antibody of claim 14, wherein the heavy chain constant region comprises a hinge region, a CH1 region, a CH2 region, and/or a CH3 region.

16. The antibody of any one of claims 14-15, wherein the heavy chain constant region comprises one or more knob-in-hole (KiH) mutations.

17. The antibody of any one of claims 14-16, wherein the heavy chain constant region comprises one or more silencing mutations.

18. The antibody of any one of claims 14-17, wherein the heavy chain constant region comprises one or more FcRn binding mutations.

19. The antibody of claim 18, wherein the one or more FcRn binding mutations comprise a T250Q mutation, an M428L mutation, or both a T250Q and an M428L mutation.

20. The antibody of any one of claims 14-19, wherein the heavy chain constant region comprises one or more protein A binding mutations.

21. The antibody of claim 20, wherein the one or more protein A binding mutations comprise an H435R mutation, a Y436F mutation, or both an H435R and a Y436F mutation.

22. The antibody of any one of claims 1-21, further comprising a light chain constant region.

23. The antibody of claim 22, wherein the light chain constant region comprises a CL region.

24. The antibody of any one of claims 1-23, which is monospecific.

25. The antibody of any one of claims 1-23, which is multispecific.

26. The antibody of claim 25, which is bispecific.

27. The antibody of claim 25 or 26, further comprising a second binding unit that binds to CD38.

28. The antibody of claim 27, wherein the second binding unit comprises:
a heavy chain variable region comprising:
(a) a CDRH1 sequence of SEQ ID NO: 43; and/or
(b) a CDRH2 sequence of any one of SEQ ID NOs: 44-45; and/or
(c) a CDRH3 sequence of SEQ ID NO: 46; and
a light chain variable region comprising:
(d) a CDRL1 sequence of SEQ ID NO: 47; and/or
(e) a CDRL2 sequence of SEQ ID NO: 48; and/or
(f) a CDRL3 sequence of SEQ ID NO: 49.

29. The antibody of claim 28, wherein the CDRH1, CDRH2 and CDRH3 sequences of the second binding unit are present in a human VH framework.

30. The antibody of claim 28 or 29, wherein the CDRL1, CDRL2 and CDRL3 sequences of the second binding unit are present in a human VL framework.

31. The antibody of any one of claims 28-30, wherein the second binding unit comprises:
a heavy chain variable region comprising:
(a) a CDRH1 sequence of SEQ ID NO: 43; and
(b) a CDRH2 sequence of any one of SEQ ID NOs: 44-45; and
(c) a CDRH3 sequence of SEQ ID NO: 46.

32. The antibody of claim 31, wherein the second binding unit comprises:
a heavy chain variable region comprising:
(a) a CDRH1 sequence of SEQ ID NO: 43, a heavy chain CDRH2 sequence of SEQ ID NO: 44, and a heavy chain CDRH3 sequence of SEQ ID NO: 46; or
(b) a CDRH1 sequence of SEQ ID NO: 43, a CDRH2 sequence of SEQ ID NO: 45, and a CDRH3 sequence of SEQ ID NO: 46.

33. The antibody of any one of claims 28-32, wherein the second binding unit comprises:
a light chain variable region comprising: a CDRL1 sequence of SEQ ID NO: 47; a CDRL2 sequence of SEQ ID NO: 48; and a CDRL3 sequence of SEQ ID NO: 49.

34. The antibody of any one of claims 28-33, wherein the second binding unit comprises:
(a) a heavy chain variable region comprising:
a CDRH1 sequence of SEQ ID NO: 43, a CDRH2 sequence of SEQ ID NO: 44, and a CDRH3 sequence of SEQ ID NO: 46; and
a light chain variable region comprising:
a CDRL1 sequence of SEQ ID NO: 47, a CDRL2 sequence of SEQ ID NO: 48, and a CDRL3 sequence of SEQ ID NO: 49; or
(b) a heavy chain variable region comprising:
a CDRH1 sequence of SEQ ID NO: 43, a CDRH2 sequence of SEQ ID NO: 45, and a CDRH3 sequence of SEQ ID NO: 46; and
a light chain variable region comprising:
a CDRL1 sequence of SEQ ID NO: 47, a CDRL2 sequence of SEQ ID NO: 48, and a CDRL3 sequence of SEQ ID NO: 49.

35. The antibody of claim 34, wherein the second binding unit comprises a heavy chain variable region sequence having at least 95% identity to any one of SEQ ID NOs: 50-51.

36. The antibody of claim 34, wherein the second binding unit comprises a heavy chain variable region sequence comprising any one of SEQ ID NOs: 50-51.

37. The antibody of any one of claims 34-35, wherein the second binding unit comprises a light chain variable region sequence having at least 95% identity to any one of SEQ ID NOs: 52-53.

38. The antibody of any one of claims 34-35, wherein the second binding unit comprises a light chain variable region sequence comprising any one of SEQ ID NOs: 52-53.

39. The antibody of any one of claims 34-35, wherein the second binding unit comprises:
(a) a heavy chain variable region sequence of SEQ ID NO: 50 and a light chain variable region sequence of SEQ ID NO: 52; or
(b) a heavy chain variable region sequence of SEQ ID NO: 51 and a light chain variable region sequence of SEQ ID NO: 53.

40. An antibody that binds to LY6G6D and CD3ε, comprising:
a first light chain subunit comprising SEQ ID NO: 57;
a first heavy chain subunit comprising SEQ ID NO: 54;
a second light chain subunit comprising SEQ ID NO: 57; and
a second heavy chain subunit comprising SEQ ID NO: 56.

41. An antibody that binds to LY6G6D and CD3ε, comprising:
a first light chain subunit comprising SEQ ID NO: 57;
a first heavy chain subunit comprising SEQ ID NO: 55;
a second light chain subunit comprising SEQ ID NO: 57; and
a second heavy chain subunit comprising SEQ ID NO: 56.

42. An antibody that binds to LY6G6D and CD3ε, comprising:
a first light chain subunit comprising SEQ ID NO: 57;
a first heavy chain subunit comprising SEQ ID NO: 58;
a second light chain subunit comprising SEQ ID NO: 57; and
a second heavy chain subunit comprising SEQ ID NO: 60.

43. An antibody that binds to LY6G6D and CD3ε, comprising:
a first light chain subunit comprising SEQ ID NO: 57;
a first heavy chain subunit comprising SEQ ID NO: 59;
a second light chain subunit comprising SEQ ID NO: 57; and
a second heavy chain subunit comprising SEQ ID NO: 60.

44. An antibody that binds to LY6G6D and CD3ε, comprising:
a first light chain subunit comprising SEQ ID NO: 57;
a first heavy chain subunit comprising SEQ ID NO: 61;
a second light chain subunit comprising SEQ ID NO: 57; and
a second heavy chain subunit comprising SEQ ID NO: 62.

45. An antibody that binds to LY6G6D and CD3ε, comprising:
a first light chain subunit comprising SEQ ID NO: 69;
a first heavy chain subunit comprising SEQ ID NO: 64;
a second light chain subunit comprising SEQ ID NO: 69; and
a second heavy chain subunit comprising SEQ ID NO: 66.

46. An antibody that binds to LY6G6D and CD3ε, comprising:
a first light chain subunit comprising SEQ ID NO: 69;
a first heavy chain subunit comprising SEQ ID NO: 65;
a second light chain subunit comprising SEQ ID NO: 69; and
a second heavy chain subunit comprising SEQ ID NO: 66.

47. An antibody that binds to LY6G6D and CD3ε, comprising:
a first light chain subunit comprising SEQ ID NO: 69;
a first heavy chain subunit comprising SEQ ID NO: 67;
a second light chain subunit comprising SEQ ID NO: 69; and
a second heavy chain subunit comprising SEQ ID NO: 68.

48. A pharmaceutical composition comprising the antibody of any one of claims 1-47.

49. A method of treatment, comprising administering to an individual in need an effective dose of the antibody of any one of claims 1-47, or the pharmaceutical composition of claim 48.

50. A method for the treatment of a disorder characterized by expression of LY6G6D, comprising administering to a subject with said disorder the antibody of any one of claims 1-47, or the pharmaceutical composition of claim 48.

51. Use of an antibody of any one of claims 1-47, in the preparation of a medicament for the treatment of a disorder characterized by expression of LY6G6D.

52. An antibody of any one of claims 1-47, for use in the treatment of a disorder characterized by expression of LY6G6D.

53. The method, use, or antibody of any one of claims 49-52, wherein the disorder is a cancer.

54. The method, use, or antibody of claim 53, wherein the cancer is a solid tumor cancer.

55. The method, use, or antibody of claim 54, wherein the solid tumor cancer is colorectal cancer.

56. The method, use, or antibody of claim 55, wherein the colorectal cancer is refractive to chemotherapy and/or immune checkpoint inhibitor therapy.

57. A polynucleotide encoding an antibody of any one of claims 1-47.

58. A vector comprising the polynucleotide of claim 57.

59. A cell comprising the vector of claim 58.

60. A method of producing an antibody of any one of claims 1-47, comprising growing a cell according to claim 59 under conditions permissive for expression of the antibody, and isolating the antibody.

61. A kit comprising the antibody of any one of claims 1-47, or the pharmaceutical composition of claim 48, and instructions for use.

62. The kit of claim 61, further comprising an additional therapeutic agent.

63. The kit of claim 62, wherein the additional therapeutic agent comprises a chemotherapeutic drug.

64. A diagnostic method for determining whether a subject has or is at risk of developing a disorder characterized by expression of LY6G6D, the method comprising:
contacting a biological test sample from the subject with an antibody that binds to LY6G6D to generate an LY6G6D-antibody complex, wherein the antibody comprises:
(a) a heavy chain variable region comprising:
a CDRH1 sequence of SEQ ID NO: 1, a CDRH2 sequence of SEQ ID NO: 7, and a CDRH3 sequence of SEQ ID NO: 12; and
a light chain variable region comprising:
a CDRL1 sequence of SEQ ID NO: 16, a CDRL2 sequence of SEQ ID NO: 20, and a CDRL3 sequence of SEQ ID NO: 24; or
(b) a heavy chain variable region comprising:
a CDRH1 sequence of SEQ ID NO: 2, a CDRH2 sequence of SEQ ID NO: 8, and a CDRH3 sequence of SEQ ID NO: 13; and
a light chain variable region comprising:
a CDRL1 sequence of SEQ ID NO: 17, a CDRL2 sequence of SEQ ID NO: 21, and a CDRL3 sequence of SEQ ID NO: 25; or
(c) a heavy chain variable region comprising:
a CDRH1 sequence of SEQ ID NO: 3, a CDRH2 sequence of SEQ ID NO: 9, and a CDRH3 sequence of SEQ ID NO: 13; and
a light chain variable region comprising:
a CDRL1 sequence of SEQ ID NO: 17, a CDRL2 sequence of SEQ ID NO: 21, and a CDRL3 sequence of SEQ ID NO: 25; or
(d) a heavy chain variable region comprising:
a CDRH1 sequence of SEQ ID NO: 4, a CDRH2 sequence of SEQ ID NO: 10, and a CDRH3 sequence of SEQ ID NO: 14; and
a light chain variable region comprising:
a CDRL1 sequence of SEQ ID NO: 18, a CDRL2 sequence of SEQ ID NO: 22, and a CDRL3 sequence of SEQ ID NO: 24; or
(e) a heavy chain variable region comprising:
a CDRH1 sequence of SEQ ID NO: 5, a CDRH2 sequence of SEQ ID NO: 11, and a CDRH3 sequence of SEQ ID NO: 15; and
a light chain variable region comprising:
a CDRL1 sequence of SEQ ID NO: 19, a CDRL2 sequence of SEQ ID NO: 23, and a CDRL3 sequence of SEQ ID NO: 26; or
(f) a heavy chain variable region comprising:
a CDRH1 sequence of SEQ ID NO: 6, a CDRH2 sequence of SEQ ID NO: 11, and a CDRH3 sequence of SEQ ID NO: 15; and
a light chain variable region comprising:
a CDRL1 sequence of SEQ ID NO: 19, a CDRL2 sequence of SEQ ID NO: 23, and a CDRL3 sequence of SEQ ID NO: 26;
detecting a concentration of the LY6G6D-antibody complex in the biological test sample; and
comparing the concentration of the LY6G6D-antibody complex to a reference value to determine whether the subject has or is at risk of developing the disorder.

65. The diagnostic method of claim 64, wherein the disorder is a cancer.

66. The diagnostic method of claim 65, wherein the cancer is a solid tumor cancer.

67. The diagnostic method of claim 66, wherein the solid tumor cancer is colorectal cancer.


SEQUENCE LISTING (88 sequences)

SEQ ID NO: 1 - protein, 5 aa, synthetic construct
GFWMS

SEQ ID NO: 2 - protein, 5 aa, synthetic construct
SYSMH

SEQ ID NO: 3 - protein, 5 aa, synthetic construct
SYSIH

SEQ ID NO: 4 - protein, 5 aa, synthetic construct
DFWMS

SEQ ID NO: 5 - protein, 5 aa, synthetic construct
SYGIT

SEQ ID NO: 6 - protein, 5 aa, synthetic construct
SYGIL

SEQ ID NO: 7 - protein, 17 aa, synthetic construct
DIDSSGSVRNYAPSIKD

SEQ ID NO: 8 - protein, 17 aa, synthetic construct
RIDPISGGTRYNEMFKN

SEQ ID NO: 9 - protein, 17 aa, synthetic construct
RIDPISGGTLYNEMFKN

SEQ ID NO: 10 - protein, 17 aa, synthetic construct
DINSDGSAINYAPSLKD

SEQ ID NO: 11 - protein, 16 aa, synthetic construct
VVWPSGATNYNSALNS

SEQ ID NO: 12 - protein, 4 aa, synthetic construct
AMDY

SEQ ID NO: 13 - protein, 7 aa, synthetic construct
FTMLVDH

SEQ ID NO: 14 - protein, 4 aa, synthetic construct
SLDY

SEQ ID NO: 15 - skipped

SEQ ID NO: 16 - protein, 16 aa, synthetic construct
RSSQSIVHRDGNTYLA

SEQ ID NO: 17 - protein, 16 aa, synthetic construct
KSSQSLLYSDGRTYLN

SEQ ID NO: 18 - protein, 16 aa, synthetic construct
RSSQSIVHSNGNTFLA

SEQ ID NO: 19 - protein, 16 aa, synthetic construct
RSSKSLLHSNGNTYLY

SEQ ID NO: 20 - protein, 7 aa, synthetic construct
KLSYRFS

SEQ ID NO: 21 - protein, 7 aa, synthetic construct
LVSKLDS

SEQ ID NO: 22 - protein, 7 aa, synthetic construct
KVSNRFS

SEQ ID NO: 23 - protein, 7 aa, synthetic construct
RVSNLAS

SEQ ID NO: 24 - protein, 9 aa, synthetic construct
LQGSHIPLT

SEQ ID NO: 25 - protein, 9 aa, synthetic construct
WQGTHFPQT

SEQ ID NO: 26 - protein, 9 aa, synthetic construct
MQHLEYPYT

SEQ ID NO: 27 - protein, 113 aa, synthetic construct
EVQLLETGGGLVQPGGSRRLSCEGSGFTFSGFWMSWVRQTPGKTLEWIGDIDSSGSVRNYAPSIKDRFTFFRDNDKNTLYLQMSNVRSEDTATYFCLRAMDYWGHGTSVTVSS

SEQ ID NO: 28 - protein, 116 aa, synthetic construct
QVQLQQPGAELVKPGTSVKLSCKASGYTFTSYSMHWVKQRPGRGLEWIGRIDPISGGTRYNEMFKNKATLTVDVPSSTAYMQLNSLTSEDSAVYYCARFTMLVDHWGQGTTLTVSS

SEQ ID NO: 29 - protein, 116 aa, synthetic construct
EVQLVQSGAEVKKPGASVKVSCKASGYTFTSYSMHWVRQAPGQGLEWIGRIDPISGGTRYNEMFKNWATLTVDVSISTAYMELSRLRSDDTAVYYCARFTMLVDHWGQGTTVTVSS

SEQ ID NO: 30 - protein, 116 aa, synthetic construct
EVQLVQSGAEVKKPGASVKVSCKASGYTFTSYSIHWVRQAPGQGLEWIGRIDPISGGTLYNEMFKNWATLTVDVSISTAYMELSRLRSDDTAVYYCARFTMLVDHWGQGTTVTVSS

SEQ ID NO: 31 - protein, 113 aa, synthetic construct
EVQLLETGGGPVQPGGSRGLSCEGSGFTFSDFWMSWVRQTPGKTLEWIGDINSDGSAINYAPSLKDRFTIFRDNDKSTLHLQMSNVRSEDTATYFCVKSLDYWGQGISVTVSS

SEQ ID NO: 32 - protein, 111 aa, synthetic construct
QVQLKESGPGLVAPSQSLSITCTVSGFSLTSYGITWVRQPPGKGLEWLGVVWPSGATNYNSALNSRLSISKDNSKSQVFLKMNSLQTDDTARYYCARWDYWGQGTTLTVSS

SEQ ID NO: 33 - protein, 111 aa, synthetic construct
EVQLVQSGAEVKKPGASVKVSCKVSGFSLTSYGITWVRQAPGQGLEWLGVVWPSGATNYNSALNSRLTITKDNSTSTVYMELRSLRSDDTAVYYCARWDYWGQGTTVTVSS

SEQ ID NO: 34 - protein, 111 aa, synthetic construct
EVQLVQSGAEVKKPGASVKVSCKVSGFSLTSYGILWVRQAPGQGLEWLGVVWPSGATNYNSALNSRLTITKDTSTSTVYMELRSLRSDDTAVYYCARWDYWGQGTTVTVSS

SEQ ID NO: 35 - protein, 112 aa, synthetic construct
EVLMTQTPLSLPVSLGDQASISCRSSQSIVHRDGNTYLAWYLQKPGQSPKLLIYKLSYRFSGVPDRFSGSGSGTDFTLRISRVEAEDLGLYYCLQGSHIPLTFGAGTKLELK

SEQ ID NO: 36 - protein, 112 aa, synthetic construct
DVVMTQTPLTLSVTIGQPASISCKSSQSLLYSDGRTYLNWMLQRPGQSPKRLIYLVSKLDSGVPDRFTGSGSGTDFTLKISRVEAEDLGIYYCWQGTHFPQTFGGGTKLEIK

SEQ ID NO: 37 - protein, 112 aa, synthetic construct
DVVMTQSPLSLPVTLGQPASISCKSSQSLLYSDGRTYLNWMQQRPGQSPRRLIYLVSKLDSGVPDRFSGSGSGTDFTLKISRVEAEDVGVYYCWQGTHFPQTFGGGTKVEIK

SEQ ID NO: 38 - protein, 112 aa, synthetic construct; identical to SEQ ID NO: 37

SEQ ID NO: 39 - protein, 112 aa, synthetic construct
DILMTQNPLSLSVSLGDQASISCRSSQSIVHSNGNTFLAWYLQKPGQSPKLLIYKVSNRFSGVPDRFSGSGSGTDFTLRISRVEAEDLGIYYCLQGSHIPLTFGAGTTLELK

SEQ ID NO: 40 - protein, 112 aa, synthetic construct
DIVMTQAAPSVPVTPGESVSISCRSSKSLLHSNGNTYLYWFLQRPGQSPQLLIYRVSNLASGVPDRFGGSGSGTAFTLRISRVEAEDVGVYYCMQHLEYPYTFGGGTKLEIK

SEQ ID NO: 41 - protein, 112 aa, synthetic construct
DIVMTQSPLSLPVTPGEPASISCRSSKSLLHSNGNTYLYWFLQKPGQSPQLLIYRVSNLASGVPDRFSGSGSGTDFTLKISRVEAEDVGVYYCMQHLEYPYTFGQGTKLEIK

SEQ ID NO: 42 - protein, 112 aa, synthetic construct; identical to SEQ ID NO: 41

SEQ ID NO: 43 - protein, 5 aa, synthetic construct
TYAMN

SEQ ID NO: 44 - protein, 19 aa, synthetic construct
RIRSKYNNYATYYADSVKD

SEQ ID NO: 45 - protein, 19 aa, synthetic construct
RIRSKANNYATYYADSVKG

SEQ ID NO: 46 - protein, 14 aa, synthetic construct
HGNFGNSYVSWFAY

SEQ ID NO: 47 - protein, 14 aa, synthetic construct
GSSTGAVTTSNYAN

SEQ ID NO: 48 - protein, 7 aa, synthetic construct
GTNKRAP

SEQ ID NO: 49 - protein, 9 aa, synthetic construct
ALWYSNLWV

SEQ ID NO: 50 - protein, 125 aa, synthetic construct
EVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSS

SEQ ID NO: 51 - protein, 125 aa, synthetic construct
EVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSS

SEQ ID NO: 52 - protein, 109 aa, synthetic construct
QAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVL

SEQ ID NO: 53 - protein, 109 aa, synthetic construct
EAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVL

SEQ ID NO: 54 - protein, 705 aa, synthetic construct
EVQLVQSGAEVKKPGASVKVSCKASGYTFTSYSMHWVRQAPGQGLEWIGRIDPISGGTRYNEMFKNWATLTVDVSISTAYMELSRLRSDDTAVYYCARFTMLVDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVLGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 55 - protein, 705 aa, synthetic construct
EVQLVQSGAEVKKPGASVKVSCKASGYTFTSYSMHWVRQAPGQGLEWIGRIDPISGGTRYNEMFKNWATLTVDVSISTAYMELSRLRSDDTAVYYCARFTMLVDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 56 - protein, 445 aa, synthetic construct
EVQLVQSGAEVKKPGASVKVSCKASGYTFTSYSMHWVRQAPGQGLEWIGRIDPISGGTRYNEMFKNWATLTVDVSISTAYMELSRLRSDDTAVYYCARFTMLVDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 219 aa, synthetic construct
DVVMTQSPLSLPVTLGQPASISCKSSQSLLYSDGRTYLNWMQQRPGQSPRRLIYLVSKLDSGVPDRFSGSGSGTDFTLKISRVEAEDVGVYYCWQGTHFPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 705 aa, synthetic construct
EVQLVQSGAEVKKPGASVKVSCKASGYTFTSYSIHWVRQAPGQGLEWIGRIDPISGGTLYNEMFKNWATLTVDVSISTAYMELSRLRSDDTAVYYCARFTMLVDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVLGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 59 - protein, 705 aa, synthetic construct
EVQLVQSGAEVKKPGASVKVSCKASGYTFTSYSIHWVRQAPGQGLEWIGRIDPISGGTLYNEMFKNWATLTVDVSISTAYMELSRLRSDDTAVYYCARFTMLVDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 60 - protein, 445 aa, synthetic construct
EVQLVQSGAEVKKPGASVKVSCKASGYTFTSYSIHWVRQAPGQGLEWIGRIDPISGGTLYNEMFKNWATLTVDVSISTAYMELSRLRSDDTAVYYCARFTMLVDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 705 aa, synthetic construct
EVQLVQSGAEVKKPGASVKVSCKASGYTFTSYSIHWVRQAPGQGLEWIGRIDPISGGTLYNEMFKNWATLTVDVSISTAYMELSRLRSDDTAVYYCARFTMLVDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLGGGGSDKTHTCPPCPAPEAAGAPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRFTQKSLSLSPG

SEQ ID NO: 62 - protein, 445 aa, synthetic construct
EVQLVQSGAEVKKPGASVKVSCKASGYTFTSYSIHWVRQAPGQGLEWIGRIDPISGGTLYNEMFKNWATLTVDVSISTAYMELSRLRSDDTAVYYCARFTMLVDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPG

SEQ ID NO: 63 - protein, 219 aa, synthetic construct; identical to SEQ ID NO: 57

SEQ ID NO: 64 - protein, 700 aa, synthetic construct
EVQLVQSGAEVKKPGASVKVSCKVSGFSLTSYGILWVRQAPGQGLEWLGVVWPSGATNYNSALNSRLTITKDTSTSTVYMELRSLRSDDTAVYYCARWDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVLGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 65 - protein, 700 aa, synthetic construct
EVQLVQSGAEVKKPGASVKVSCKVSGFSLTSYGILWVRQAPGQGLEWLGVVWPSGATNYNSALNSRLTITKDTSTSTVYMELRSLRSDDTAVYYCARWDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 66 - protein, 440 aa, synthetic construct
EVQLVQSGAEVKKPGASVKVSCKVSGFSLTSYGILWVRQAPGQGLEWLGVVWPSGATNYNSALNSRLTITKDTSTSTVYMELRSLRSDDTAVYYCARWDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 700 aa, synthetic construct
EVQLVQSGAEVKKPGASVKVSCKVSGFSLTSYGILWVRQAPGQGLEWLGVVWPSGATNYNSALNSRLTITKDTSTSTVYMELRSLRSDDTAVYYCARWDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVLGGGGSDKTHTCPPCPAPEAAGAPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRFTQKSLSLSPG

SEQ ID NO: 68 - protein, 440 aa, synthetic construct
EVQLVQSGAEVKKPGASVKVSCKVSGFSLTSYGILWVRQAPGQGLEWLGVVWPSGATNYNSALNSRLTITKDTSTSTVYMELRSLRSDDTAVYYCARWDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPG

SEQ ID NO: 69 - protein, 219 aa, synthetic construct
DIVMTQSPLSLPVTPGEPASISCRSSKSLLHSNGNTYLYWFLQKPGQSPQLLIYRVSNLASGVPDRFSGSGSGTDFTLKISRVEAEDVGVYYCMQHLEYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70-75 - skipped (6 entries)

SEQ ID NO: 76 - protein, 396 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLELQLEESCAEAQDGELDGLWTTITIFITLFLLSVCYSATVTFFKVKWIFSSVVDLKQTIVPDYRNMIRQGA

SEQ ID NO: 77-83 - skipped (7 entries)

SEQ ID NO: 84 - protein, 5 aa, synthetic construct
GGGGS

SEQ ID NO: 85 - protein, 10 aa, synthetic construct
GGGGSGGGGS

SEQ ID NO: 86 - protein, 10 aa, synthetic construct
EPKSCDKTHT

SEQ ID NO: 87 - protein, 10 aa, synthetic construct
EPKSSDKTHT

SEQ ID NO: 88 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS